(19) United States
(12) Patent Application Publication
(10) Pub. No.: US 2014/0065612 A1
(43) Pub. Date: Mar. 6, 2014

(54) METHOD FOR IN VITRO DETECTING KERATIN GENE FUSION OF SQUAMOUS-CELL CANCER

(71) Applicant: CHINA MEDICAL UNIVERSITY, Taichung City (TW)

(72) Inventors: FUU-JEN TSAI, Taichung City (TW); JINN-CHYUAN JIM SHEU, Taichung City (TW); JACK CHENG, Taichung City (TW); CHUN CHIN CHAO, Tainan City (TW)

(73) Assignee: CHINA MEDICAL UNIVERSITY, Taichung City (TW)

(21) Appl. No.: 13/785,829

(22) Filed: Mar. 5, 2013

(30) Foreign Application Priority Data
Sep. 5, 2012 (TW) 101132329

(51) Int. Cl. C12Q 1/68

(52) U.S. Cl. CPC C12Q 1/6886
USPC 435/6.11; 435/6.12

(57) ABSTRACT

A method for in vitro detecting keratin gene fusion of squamous-cell cancer comprises steps: (a) obtaining a sample of squamous cells from a testee; and (b) detecting whether the sample of squamous cells has gene fusion, which is likely to occur in squamous-cell cancer and unlikely to occur in healthy tissue. The sample of squamous cells is determined to have squamous-cell cancer if the gene fusion exists in the sample of squamous cells.

### FIELD OF THE INVENTION

The present invention relates to a cancer detection method, particularly to a method for in vitro detecting keratin gene fusion of squamous-cell cancer.

### BACKGROUND OF THE INVENTION

Squamous-cell cancers may occur in many regions, including skin, lip, mouth, weasand, bladder, prostate, lung, vagina, and cervix. The morbidities of different squamous-cell cancers correlate with age, sex, race, geography, and heredity. The morbidity increases with age, having a peak at the age of about 66. The males have higher morbidities of the squamous-cell cancers of the bladder and prostate than the females. The squamous-cell cancer of skin is more likely to occur in the Caucasians. The persons, who have high-dose UV exposure or have degenerative skin diseases (such as scars or ulcers), are also more likely to have skin squamous-cell cancers. The persons, who contact arsenic or other industrial pollutants, have higher risk of squamous-cell cancers.

At present, the over-expression of genes, in cooperation with IHC (immunohistochemical) staining, is usually used to diagnose squamous-cell cancers. SNB (Sentinel Node Biopsy) is normally used to screen the testees, and then the suspected cases are verified with IHC staining. The over-expression of genes—VEGF-A, VEGF-C, EGFR, COX-2, c-myc, Cyclin D1, Cyclin A, Rb, p16, p21, p27, and p34—are usually used as an auxiliary of squamous-cell cancer diagnosis, referring to a paper by Seki, et al., 2011, Oral Oncol., 47(7):588-93; a paper by Massano, et al., 2006, Oral Surg Oral Med Oral Pathol Oral Radiol Endod, pp. 67-76; and a paper by Alkureishi, et al., 2009, Ann Surg Oncol., 16(11):3190-210. The abovementioned gene markers are not expressed obviously in the early stage of cancers but expressed significantly in the later stage. The abovementioned gene markers are hard to distinguish abnormal cells from normal cells in the early stage and likely to cause false negative errors. Therefore, the genetic method to detect squamous-cell cancers still has room to improve.

One target of oncological research is to find out the genes related with the initiation, growth and spread of cancers. Several types of cellular mutations have been found to relate with cancers, including substitution, insertion, deletion and translocation of base groups, and variation of the copy number. More and more researches show that chromosome translocation correlates with cancers (refer to a paper by Rowley, Nat Rev Cancer 1: 245 (2001)). However, the cases of chromosome translocations found in epithelial tumors, which contribute much to the morbidity and mortality of human cancers, are less than 1% of the known cases of the chromosome translocations (refer to a paper by Mitelman, Mutat Res 462: 247 (2000)).

The present invention discloses a method for detecting the gene fusion correlating with squamous-cell cancers, providing a new way to detect, research, and treat squamous-cell cancers.

### SUMMARY OF THE INVENTION

The primary objective of the present invention is to provide a method for detecting keratin gene fusion, which is a new label of squamous-cell cancer, to promote the accuracy of squamous-cell cancer diagnosis, whereby is overcome the false negative errors occurring in the conventional squamous-cell cancer detection method.

To achieve the abovementioned objective, the present invention proposes a first method for in vitro detecting keratin gene fusion of squamous-cell cancer, which comprises steps: (a) obtaining a squamous-cell sample; and (b) detecting whether gene fusion occurs in the squamous-cell sample, wherein the gene fusion includes a 5′ terminal having a type I keratin gene and a 3′ terminal having a type II keratin gene, a DSP gene, an MYH9 gene, or an SFN gene, and wherein the squamous-cell sample is determined to have squamous-cell cancer if the gene fusion exists in the squamous-cell sample.

In one embodiment of the first method, Step (b) includes detecting whether the squamous-cell sample has chromosome translocation in genomic DNA, wherein the sequence of the genomic DNA includes a 5′ terminal having a type I keratin gene, and a 3′ terminal having a type II keratin gene, a DSP gene, an MYH9 gene or an SFN gene.

In one embodiment of the first method, Step (b) includes detecting whether the squamous-cell sample has mRNA transcript of gene fusion, wherein the mRNA transcript of gene fusion includes a 3′ terminal transcripted from a type I keratin gene, and a 5′ terminal transcripted from a type II keratin gene, a DSP gene, an MYH9 gene or an SFN gene.

In one embodiment of the first method, the type I keratin is selected from a group consisting of genes of KRT14, KRT16, KRT17, KRT18, KRT19, and KRT20; the type II keratin is selected from a group consisting of genes of KRT6A, KRT6B, KRT6C, KRT5, KRT7, and KRT8. The KRT14 gene has a nucleotide sequence SEQ ID No: 15. The KRT16 gene has a nucleotide sequence SEQ ID No: 16. The KRT17 gene has a nucleotide sequence SEQ ID No: 17. The KRT18 gene has a nucleotide sequence SEQ ID No: 35. The KRT19 gene has a nucleotide sequence SEQ ID No: 37. The KRT20 gene has a nucleotide sequence SEQ ID No: 39. The KRT6A gene has a nucleotide sequence SEQ ID No: 11. The KRT6B gene has a nucleotide sequence SEQ ID No: 12. The KRT6C gene has a nucleotide sequence SEQ ID No: 13. The KRT5 gene has a nucleotide sequence SEQ ID No: 14. The KRT7 gene has a nucleotide sequence SEQ ID No: 31. The KRT8 gene has a nucleotide sequence SEQ ID No: 33. The DSP gene has a nucleotide sequence SEQ ID No: 25. The MYH9 gene has a nucleotide sequence SEQ ID No: 26. The SFN gene has a nucleotide sequence SEQ ID No: 27.

In one embodiment of the first method, Step (b) includes detecting whether the squamous-cell sample has a gene fusion protein, wherein the gene fusion protein includes an N terminal having the amino acid sequence of a type I keratin, and a C terminal having the amino acid sequence of a type II keratin, a DSP protein, an MYH9 protein or an SFN protein.

In one embodiment of the first method, the amino acid sequence of the type I keratin is selected from a group consisting of amino acid sequences of a KRT14 protein, a KRT16 protein, a KRT17 protein, a KRT18 protein, a KRT19 protein, and a KRT20 protein; the amino acid sequence of the type II keratin is selected from a group consisting of amino acid sequences of a KRT6A protein, a KRT6B protein, a KRT6C protein, a KRT5 protein, a KRT7 protein, and a KRT8 protein. The amino acid sequence of the KRT14 protein has an amino acid sequence SEQ ID No: 22. The amino acid sequence of the KRT16 protein has an amino acid sequence SEQ ID No: 23. The amino acid sequence of the KRT17 protein has an amino acid sequence SEQ ID No: 24. The amino acid sequence of the KRT18 protein has an amino acid sequence SEQ ID No: 36. The amino acid sequence of the KRT19 protein has an amino acid sequence SEQ ID No: 38. The amino acid sequence of the KRT20 protein has an amino acid sequence SEQ ID No: 40. The amino acid sequence of the KRT6A protein has an amino acid sequence SEQ ID No: 18. The amino acid sequence of the KRT6B protein has an amino acid sequence SEQ ID No: 19. The amino acid sequence of the KRT6C protein has an amino acid sequence SEQ ID No: 20. The amino acid sequence of the KRT5 protein has an amino acid sequence SEQ ID No: 21. The amino acid sequence of the KRT7 protein has an amino acid sequence SEQ ID No: 32. The amino acid sequence of the KRT8 protein has an amino acid sequence SEQ ID No: 34. The amino acid sequence of the DSP protein has an amino acid sequence SEQ ID No: 28. The amino acid sequence of the MYH9 protein has an amino acid sequence SEQ ID No: 29. The amino acid sequence of the SFN protein has an amino acid sequence SEQ ID No: 30.

In one embodiment of the first method, the squamous-cell sample is selected from a group consisting of oral epithelial cells, cervical epithelial cells, nasopharyngeal epithelial cells and esophageal epithelial cells.

The present invention further proposes a second method for in vitro detecting keratin gene fusion of squamous-cell cancer, which comprises steps: (a) obtaining a squamous-cell sample; and (b) detecting whether gene fusion occurs in the squamous-cell sample, wherein the gene fusion includes a 5′ terminal having a type II keratin gene and a 3′ terminal having a type I keratin gene, a DSP gene, an MYH9 gene, or an SFN gene, and wherein the squamous-cell sample is determined to have squamous-cell cancer if the gene fusion exists in the squamous-cell sample.

In one embodiment of the second method, Step (b) includes detecting whether the squamous-cell sample has chromosome translocation in genomic DNA, wherein the sequence of the genomic DNA includes a 5′ terminal having a type II keratin gene, and a 3′ terminal having a type I keratin gene, a DSP gene, an MYH9 gene or an SFN gene.

In one embodiment of the second method, Step (b) includes detecting whether the squamous-cell sample has mRNA transcript of gene fusion, wherein the mRNA transcript of gene fusion includes a 3′ terminal transcripted from a type II keratin gene, and a 5′ terminal transcripted from a type I keratin gene, a DSP gene, an MYH9 gene or an SFN gene.

In one embodiment of the second method, the type I keratin is selected from a group consisting of genes of KRT14, KRT16, KRT17, KRT18, KRT19, and KRT20; the type II keratin is selected from a group consisting of genes of KRT6A, KRT6B, KRT6C, KRT5, KRT7, and KRT8. The KRT14 gene has a nucleotide sequence SEQ ID No: 15. The KRT16 gene has a nucleotide sequence SEQ ID No: 16. The KRT17 gene has a nucleotide sequence SEQ ID No: 17. The KRT18 gene has a nucleotide sequence SEQ ID No: 35. The KRT19 gene has a nucleotide sequence SEQ ID No: 37. The KRT20 gene has a nucleotide sequence SEQ ID No: 39. The KRT6A gene has a nucleotide sequence SEQ ID No: 11. The KRT6B gene has a nucleotide sequence SEQ ID No: 12. The KRT6C gene has a nucleotide sequence SEQ ID No: 13. The KRT5 gene has a nucleotide sequence SEQ ID No: 14. The KRT7 gene has a nucleotide sequence SEQ ID No: 31. The KRT8 gene has a nucleotide sequence SEQ ID No: 33. The DSP gene has a nucleotide sequence SEQ ID No: 25. The MYH9 gene has a nucleotide sequence SEQ ID No: 26. The SFN gene has a nucleotide sequence SEQ ID No: 27.

In one embodiment of the second method, Step (b) includes detecting whether the squamous-cell sample has a gene fusion protein, wherein the gene fusion protein includes an N terminal having the amino acid sequence of a type II keratin, and a C terminal having the amino acid sequence of a type I keratin, a DSP protein, an MYH9 protein or an SFN protein.

In one embodiment of the second method, the amino acid sequence of the type I keratin is selected from a group consisting of amino acid sequences of a KRT14 protein, a KRT16 protein, a KRT17 protein, a KRT18 protein, a KRT19 protein, and a KRT20 protein; the amino acid sequence of the type II keratin is selected from a group consisting of amino acid sequences of a KRT6A protein, a KRT6B protein, a KRT6C protein, a KRT5 protein, a KRT7 protein, and a KRT8 protein. The amino acid sequence of the KRT14 protein has an amino acid sequence SEQ ID No: 22. The amino acid sequence of the KRT16 protein has an amino acid sequence SEQ ID No: 23. The amino acid sequence of the KRT17 protein has an amino acid sequence SEQ ID No: 24. The amino acid sequence of the KRT18 protein has an amino acid sequence SEQ ID No: 36. The amino acid sequence of the KRT19 protein has an amino acid sequence SEQ ID No: 38. The amino acid sequence of the KRT20 protein has an amino acid sequence SEQ ID No: 40. The amino acid sequence of the KRT6A protein has an amino acid sequence SEQ ID No: 18. The amino acid sequence of the KRT6B protein has an amino acid sequence SEQ ID No: 19. The amino acid sequence of the KRT6C protein has an amino acid sequence SEQ ID No: 20. The amino acid sequence of the KRT5 protein has an amino acid sequence SEQ ID No: 21. The amino acid sequence of the KRT7 protein has an amino acid sequence SEQ ID No: 32. The amino acid sequence of the KRT8 protein has an amino acid sequence SEQ ID No: 34. The amino acid sequence of the DSP protein has an amino acid sequence SEQ ID No: 28. The amino acid sequence of the MYH9 protein has an amino acid sequence SEQ ID No: 29. The amino acid sequence of the SFN protein has an amino acid sequence SEQ ID No: 30.

In one embodiment of the second method, the squamous-cell sample is selected from a group consisting of oral epithelial cells, cervical epithelial cells, nasopharyngeal epithelial cells and esophageal epithelial cells.

The present invention further proposes a third method for in vitro detecting keratin gene fusion of squamous-cell cancer, which comprises steps: (a) obtaining a squamous-cell sample; and (b) detecting whether gene fusion occurs in the squamous-cell sample, wherein the gene fusion includes a 5′ terminal having a DSP gene, an MYH9 gene, or an SFN gene and a 3′ terminal having a type I keratin gene or a type II keratin gene, and wherein the squamous-cell sample is determined to have squamous-cell cancer if the gene fusion exists in the squamous-cell sample.

In one embodiment of the third method, Step (b) includes detecting whether the squamous-cell sample has chromosome translocation in genomic DNA, wherein the sequence of the genomic DNA includes a 5′ terminal having a DSP gene, an MYH9 gene or an SFN gene, and a 3′ terminal having a type I keratin gene or a type II keratin gene.

In one embodiment of the third method, Step (b) includes detecting whether the squamous-cell sample has mRNA transcript of gene fusion, wherein the mRNA transcript of gene fusion includes a 3′ terminal transcripted from a DSP gene, an MYH9 gene or an SFN gene, and a 5′ terminal transcripted from a type I keratin gene or a type II keratin gene.

In one embodiment of the third method, the type I keratin is selected from a group consisting of genes of KRT14, KRT16, KRT17, KRT18, KRT19, and KRT20; the type II keratin is selected from a group consisting of genes of KRT6A, KRT6B, KRT6C, KRT5, KRT7, and KRT8. The KRT14 gene has a nucleotide sequence SEQ ID No: 15. The KRT16 gene has a nucleotide sequence SEQ ID No: 16. The KRT17 gene has a nucleotide sequence SEQ ID No: 17. The KRT18 gene has a nucleotide sequence SEQ ID No: 35. The KRT19 gene has a nucleotide sequence SEQ ID No: 37. The KRT20 gene has a nucleotide sequence SEQ ID No: 39. The KRT6A gene has a nucleotide sequence SEQ ID No: 11. The KRT6B gene has a nucleotide sequence SEQ ID No: 12. The KRT6C gene has a nucleotide sequence SEQ ID No: 13. The KRT5 gene has a nucleotide sequence SEQ ID No: 14. The KRT7 gene has a nucleotide sequence SEQ ID No: 31. The KRT8 gene has a nucleotide sequence SEQ ID No: 33. The DSP gene has a nucleotide sequence SEQ ID No: 25. The MYH9 gene has a nucleotide sequence SEQ ID No: 26. The SFN gene has a nucleotide sequence SEQ ID No: 27.

In one embodiment of the third method, Step (b) includes detecting whether the squamous-cell sample has a gene fusion protein, wherein the gene fusion protein includes an N terminal having the amino acid sequence of a DSP protein, an MYH9 protein or an SFN protein, and a C terminal having the amino acid sequence of a type I keratin or a type II keratin.

In one embodiment of the third method, the amino acid sequence of the type I keratin is selected from a group consisting of amino acid sequences of a KRT14 protein, a KRT16 protein, a KRT17 protein, a KRT18 protein, a KRT19 protein, and a KRT20 protein; the amino acid sequence of the type II keratin is selected from a group consisting of amino acid sequences of a KRT6A protein, a KRT6B protein, a KRT6C protein, a KRT5 protein, a KRT7 protein, and a KRT8 protein. The amino acid sequence of the KRT14 protein has an amino acid sequence SEQ ID No: 22. The amino acid sequence of the KRT16 protein has an amino acid sequence SEQ ID No: 23. The amino acid sequence of the KRT17 protein has an amino acid sequence SEQ ID No: 24. The amino acid sequence of the KRT18 protein has an amino acid sequence SEQ ID No: 36. The amino acid sequence of the KRT19 protein has an amino acid sequence SEQ ID No: 38. The amino acid sequence of the KRT20 protein has an amino acid sequence SEQ ID No: 40. The amino acid sequence of the KRT6A protein has an amino acid sequence SEQ ID No: 18. The amino acid sequence of the KRT6B protein has an amino acid sequence SEQ ID No: 19. The amino acid sequence of the KRT6C protein has an amino acid sequence SEQ ID No: 20. The amino acid sequence of the KRT5 protein has an amino acid sequence SEQ ID No: 21. The amino acid sequence of the KRT7 protein has an amino acid sequence SEQ ID No: 32. The amino acid sequence of the KRT8 protein has an amino acid sequence SEQ ID No: 34. The amino acid sequence of the DSP protein has an amino acid sequence SEQ ID No: 28. The amino acid sequence of the MYH9 protein has an amino acid sequence SEQ ID No: 29. The amino acid sequence of the SFN protein has an amino acid sequence SEQ ID No: 30.

In one embodiment of the third method, the squamous-cell sample is selected from a group consisting of oral epithelial cells, cervical epithelial cells, nasopharyngeal epithelial cells and esophageal epithelial cells.

The present invention uses gene fusion, which is absent in healthy cells and specific to the squamous-cell cancers, as the target of examination. The present invention examines whether the sample of the testee has the mRNA sequence, protein, or chromosome translocation of gene fusion, which are specific to squamous-cell cancer. Therefore, the present invention is a dedicated method to detect squamous-cell cancer. The healthy tissue in the sample would not interfere with the examination of the present invention. Therefore, the examination of the present invention has higher accuracy.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the results of gel electrophoresis of nested PCR of OSCC samples according to one embodiment of the present invention;

FIGS. 2-5 show the results of Sanger sequencing of KRT6: KRT14 gene fusion according to one embodiment of the present invention;

FIG. 6A shows the results of the preparation of the probes for OSCC according to one embodiment of the present invention;

FIG. 6B shows the concentration and purity of DNA in the preparation of the probes for OSCC according to one embodiment of the present invention;

FIG. 6C shows the results of the nick translation in the preparation of the probes for OSCC according to one embodiment of the present invention;

FIG. 6D shows the results of FISH undertaken in the cells free of chromosome translocation according to one embodiment of the present invention;

FIG. 6E shows the results of FISH revealing the chromosome translocation of gene fusion in OSCC SAT cell line according to one embodiment of the present invention;

FIG. 7 shows the results of gel electrophoresis of nested PCR of CSCC samples according to one embodiment of the present invention;

FIG. 8 shows the results of gel electrophoresis of nested PCR of NSCC samples according to one embodiment of the present invention; and

FIG. 9 shows the results of gel electrophoresis of nested PCR of ESCC samples according to one embodiment of the present invention.

### DETAILED DESCRIPTION OF THE PREFERRED EMBODIMENTS

The present invention provides a method for in vitro detecting keratin gene fusion of squamous-cell cancer, which comprises steps: (a) obtaining a squamous-cell sample; and (b) detecting whether gene fusion occurs in the squamous-cell sample. In one embodiment, the gene fusion includes a 5′ terminal having a type I keratin gene, and a 3′ terminal having a type II keratin gene, a DSP gene, an MYH9 gene or an SFN gene. In one embodiment, the gene fusion includes a 5′ terminal having a type II keratin gene, and a 3′ terminal having a type I keratin gene, a DSP gene, an MYH9 gene or an SFN gene. In one embodiment, the gene fusion includes a 5′ terminal having a DSP gene, an MYH9 gene or an SFN gene, and a 3′ terminal having a type I keratin gene or a type II keratin gene. The squamous-cell sample is determined to have squamous-cell cancer if the gene fusion exists in the squamous-cell sample.

The present invention examines whether gene fusion occurs in the squamous-cell sample from three aspects: DNA chromosome translocation, gene fusion mRNA transcript, and gene fusion protein.

I. Chromosome Translocation

In one embodiment, Step (b) includes detecting whether the squamous-cell sample has chromosome translocation in genomic DNA. In one embodiment, the sequence of the genomic DNA includes a 5′ terminal having a type I keratin gene, and a 3′ terminal having a type II keratin gene, a DSP gene, an MYH9 gene or an SFN gene. In one embodiment, the genomic DNA includes a 5′ terminal having a type II keratin gene, and a 3′ terminal having a type I keratin gene, a DSP gene, an MYH9 gene or an SFN gene. In one embodiment, the genomic DNA includes a 5′ terminal having a DSP gene, an MYH9 gene or an SFN gene, and a 3′ terminal having a type I keratin gene or a type II keratin gene. The present invention does not constrain the technology used to detect the genomic DNA chromosome translocation. Various technologies may be used to detect the genomic DNA chromosome translocation, which is very likely to appear in squamous-cell cancers, including nucleotide sequencing, nucleotide hybridization, and nucleotide amplification. The nucleotide sequencing technology may be but is not limited to be the NGS (Next Generation Sequencing) method or the Sanger sequencing method. The nucleotide hybridization technology may be but is not limited to be the ISH (In Situ Hybridization) method, the microarray method, the FISH (Fluorescent In Situ Hybridization) method, or the Southern blot method. The nucleotide amplification technology may be but is not limited to be the PCR (Polymerase Chain Reaction) method, the RT-PCR (Reverse Transcription Polymerase Chain Reaction) method, the TMA (Transcription-mediated Amplification) method, the LCR (Ligase Chain Reaction) method, the SDA (Strand Displacement Amplification) method, the NASBA (Nucleotide Sequence Based Amplification) method, or the CISH (Chromogenic In Situ Hybridization) method.

II. mRNA Transcript of Gene Fusion

In one embodiment, Step (b) includes detecting whether the squamous-cell sample has mRNA transcript of gene fusion. In one embodiment, the mRNA transcript of gene fusion includes a 3′ terminal transcripted from a type I keratin gene, and a 5′ terminal transcripted from a type II keratin gene, a DSP gene, an MYH9 gene or an SFN gene. In one embodiment, the mRNA transcript of gene fusion includes a 3′ terminal transcripted from a type II keratin gene, and a 5′ terminal transcripted from a type I keratin gene, a DSP gene, an MYH9 gene or an SFN gene. In one embodiment, the mRNA transcript of gene fusion includes a 3′ terminal transcripted from a DSP gene, an MYH9 gene or an SFN gene, and a 5′ terminal transcripted from a type I keratin gene or a type II keratin gene. The present invention does not constrain the technology used to detect the mRNA transcript of gene fusion. Various technologies may be used to detect the gene fusion mRNA, which is very likely to appear in squamous-cell cancers, including nucleotide sequencing, nucleotide hybridization, and nucleotide amplification. The nucleotide sequencing technology may be but is not limited to be the NGS (Next Generation Sequencing) method or the Sanger sequencing method. The nucleotide hybridization technology may be but is not limited to be the ISH (In Situ Hybridization) method, the microarray method, or the Southern blot method. The nucleotide amplification technology may be but is not limited to be the PCR (Polymerase Chain Reaction) method, the RT-PCR (Reverse Transcription Polymerase Chain Reaction) method, the TMA (Transcription-mediated Amplification) method, the LCR (Ligase Chain Reaction) method, the SDA (Strand Displacement Amplification) method, or the NASBA (Nucleotide Sequence Based Amplification) method.

III. Protein Product of Gene Fusion

In one embodiment, Step (b) includes detecting whether the squamous-cell sample has a gene fusion protein. In one embodiment, the gene fusion protein includes an N terminal having the amino acid sequence of a type I keratin, and a C terminal having the amino acid sequence of a type II keratin, a DSP protein, an MYH9 protein or an SFN protein. In one embodiment, the gene fusion protein includes an N terminal having the amino acid sequence of a type II keratin, and a C terminal having the amino acid sequence of a type I keratin, a DSP protein, an MYH9 protein or an SFN protein. In one embodiment, the gene fusion protein includes an N terminal having the amino acid sequence of a DSP protein, an MYH9 protein or an SFN protein, and a C terminal having the amino acid sequence of a type I keratin or a type II keratin. The present invention does not constrain the technology used to detect the gene fusion protein. Various technologies may be used to detect the gene fusion protein, which is very likely to appear in squamous-cell cancers, including the protein sequencing method, the immunoprecipitation method, the Western blot method, the ELISA (Enzyme-Linked ImmunoSorbent Assay) method, the immunohistochemistry method, the immunocytochemistry method, the flow cytometry method, and the immuno-PRC method.

The present invention is exemplified with different embodiments below. However, the scope of the present invention is not limited by these embodiments.

### Embodiment I

### Sequencing and Popularization Rate of Gene Fusion in Oral Squamous-Cell Cancer

A. Test Material and Test Method

The test material includes samples of oral squamous-cell cancer (n=48) and normal samples (n=4). All the samples of oral squamous-cell cancer are provided by the tissue bank of the China Medical University Hospital. The RNA of the samples is extracted with the RNeasy mini kit (Qiagen), quantified with the Nanodrop fluorescent absorption method, and analyzed with a gel-electrophoresis method. The high capacity cDNA RT kit (Applied Bioscience) is used to reverse-transcript 1 μg of RNA of each sample into cDNA. The cDNA is diluted by a 0.1×TE buffer solution to have a concentration of 50-80 ng/nl. Use 1 μL of cDNA as the template to undertake PCR with APP (Amyloid beta Precursor Protein) gene sequence (SEQ ID No: 1; SEQ ID No: 2) being the primer. Examine the products of PCR with gel-electrophoresis, and discard the APP-negative samples. Use 1 μL of cDNA taken from each APP-positive sample as the template to undertake PCR with the gene fusion sequence KRT6: KRT14 (SEQ ID No: 5; SEQ ID No: 6) being the external primer. Dilute the product of PCR with ten times of molecular-biological grade water. Use 1 μL of the diluted PCR product as the template to undertake nested-PCR with the gene fusion sequence KRT6: KRT14 (primer 132, SEQ ID No: 7; primer 216, SEQ ID No: 8) being the internal primer. Examine the product of the nested-PCR with gel-electrophoresis, and scoop out a gel region which contains the sequence to be analyzed. Use a gel retrieval kit (Qiagen) to retrieve the product. Use a pGEM-T easy kit (Promega) to clone the product to a carrier, and undertake Sanger sequencing.

B. Test Results

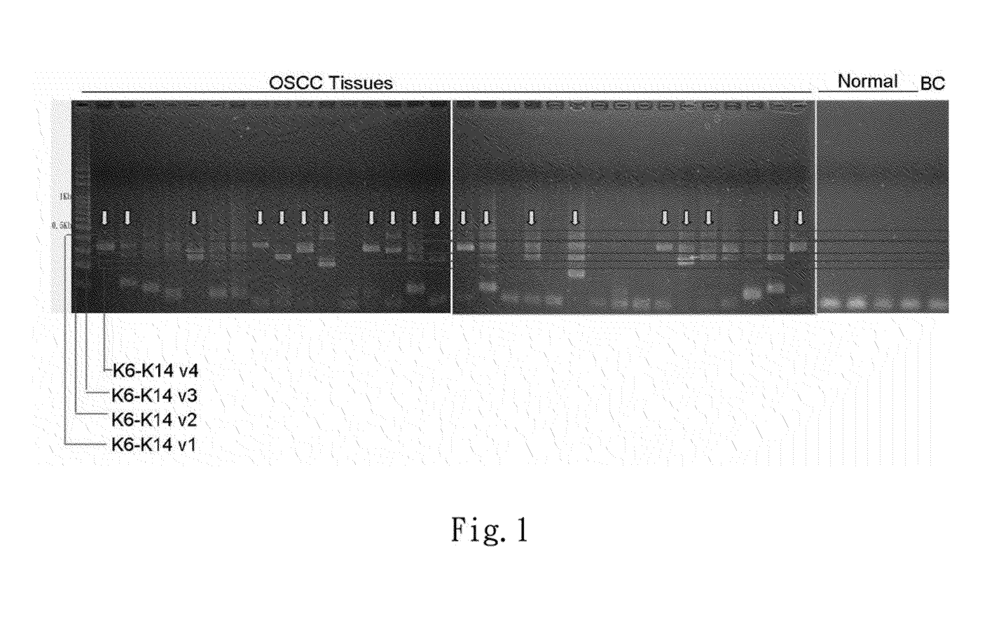

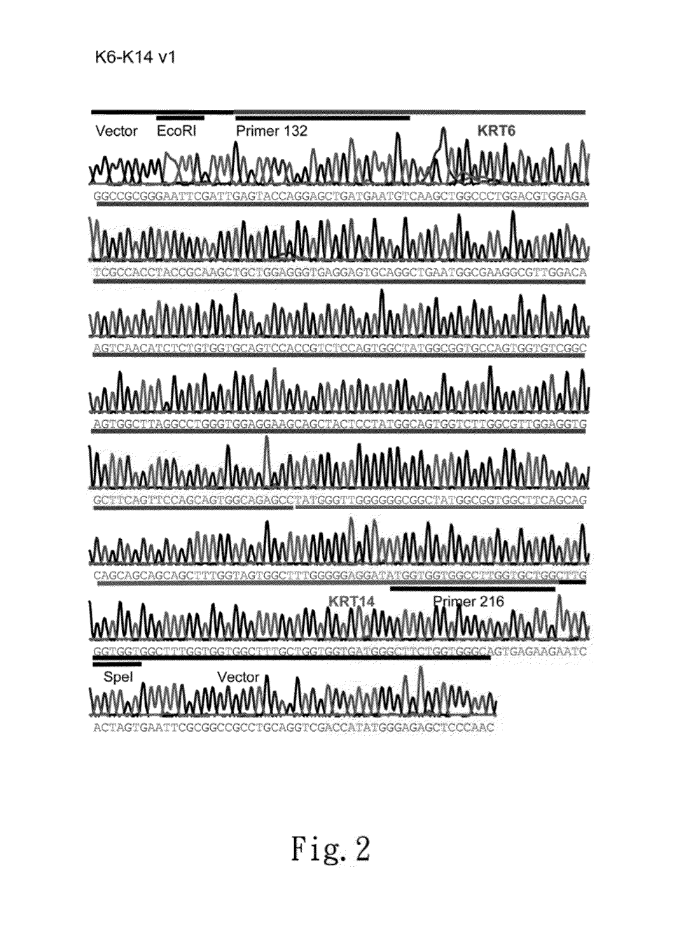

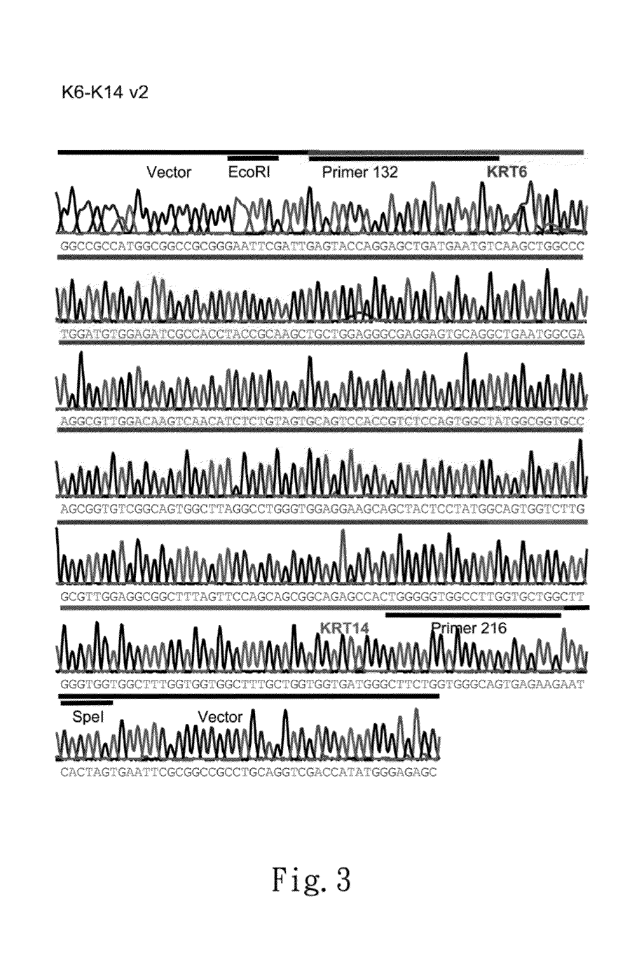

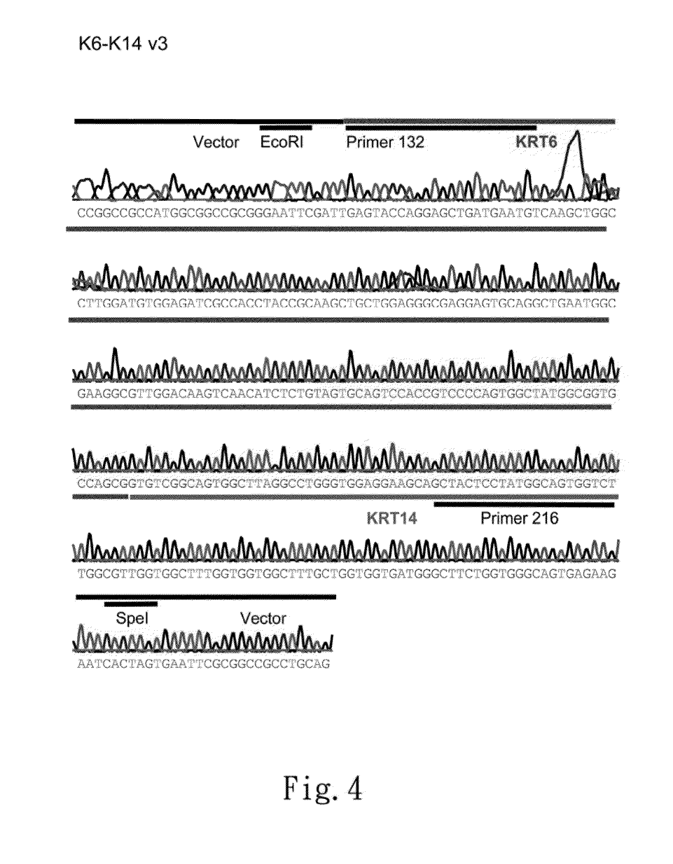

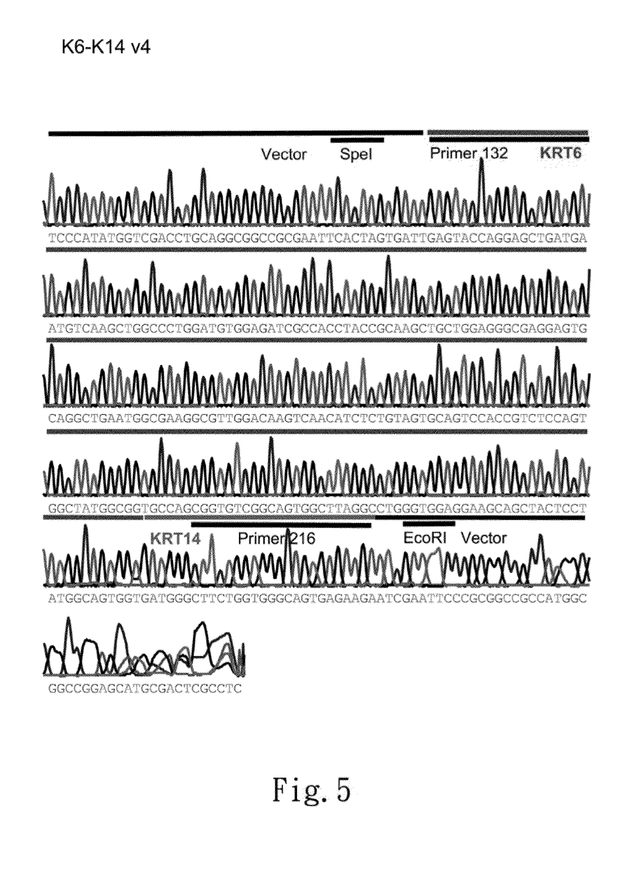

Prepare 32 samples of OSCC (Oral Squamous-Cell Cancer) tissues, 4 normal samples (normal) and 1 sample of pure water (BC) as the templates. Use the gene fusion sequences KRT6: KRT14 to undertake nested-PCR, and obtain the results shown in FIG. 1. There are 20 samples of OSCC tissues having positive reactions, which are indicated by the arrows in FIG. 1. The 4 normal samples (normal) and 1 sample of pure water (BC) have negative reactions. There are four groups of PRC products respectively having different sizes in the gel electrophoresis, which are separately designated by K6-K14 v1, K6-K14 v2, K6-K14 v3, and K6-K14 v4. The results of the Sanger sequencing of the PRC products are respectively shown in FIG. 2, FIG. 3, FIG. 4 and FIG. 5. Although the four groups of PCR products respectively have different sizes, they all belong to the KRT6: KRT14 gene fusion sequences. Therefore, the popularization rate of the KRT6: KRT14 gene fusion is 62.5% (20/32) in the OSCC samples.

### Embodiment II

FISH of the Gene Fusion of the SAT Cell Line of OSCC

A. Test Material and Test Method

a. Preparation of Sample Glasses

The test material includes the SAT cell line of OSCC. Cultivate the SAT cell line of OSCC in a T75 culture box until the cells have occupied 80% of the volume. Add 0.2 ml of EtBr (1 mg/ml) to the cells, and place them still at a temperature of 37° C. for 90 minutes. Add 0.1 ml of colcemid (Gibco) to the cells, and place them still at a temperature of 37° C. for 25 minutes. Collect and centrifugally process the cells, and remove the supernatant. Add 10 ml of 0.56% KCl to the cells, and flush them with water for 15 minutes. Centrifugally process the liquid containing cells, and remove the supernatant. Flush the cells with a solution containing methanol and glacial acetic acid by 3:1 at a temperature of 0° C. three times, and fix them. Spray the fixed cells on clean silane coating slides (Muto pure chemicals). Process the cells with 100% alcohol for 2 minutes, and process the cells with 100 μg/ml RNAseA for 60 minutes. Process the cells with 0.01N HCl containing 0.02% pepsin in a humidified box for 3 minutes, and fix the cells with 1% formaldehyde. Dehydrate the cells with 70%, 90% and 100% alcohol in sequence, and then place the cells in alcohol.

b. Preparation of Fluorescent Probes

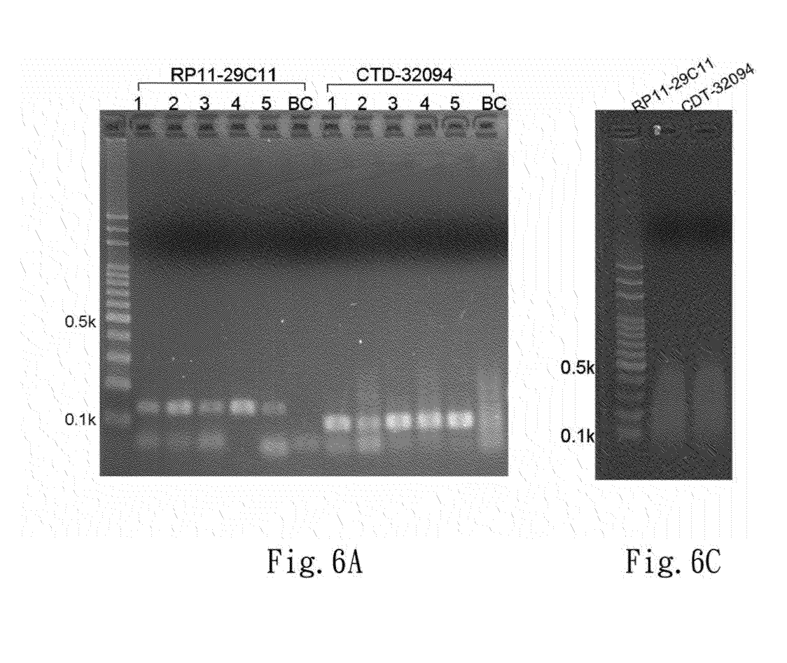

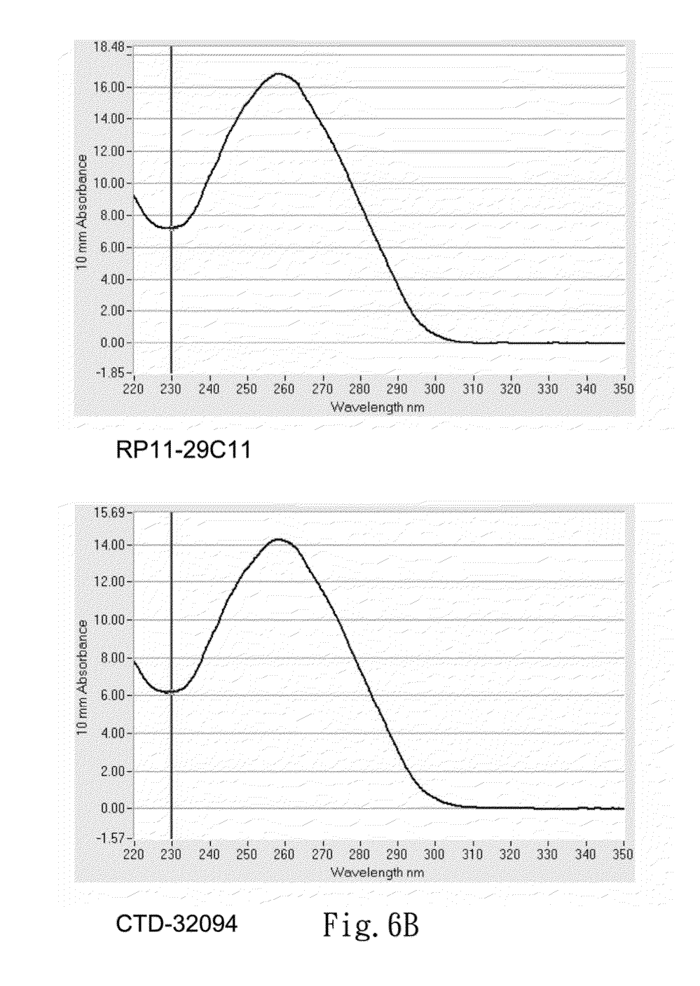

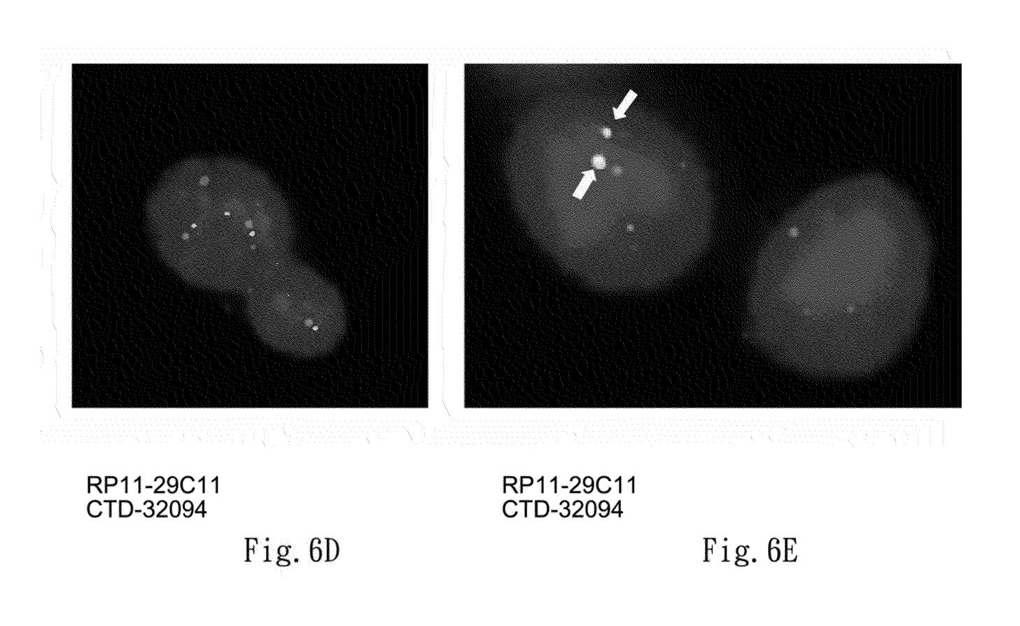

Prepare BAC clone RP11-29C11 (corresponding to the chromosome 17q21.2) and CTD-32094 (corresponding to the chromosome 12q13.13) (both are products of Invitrogen). Rub the liquid containing the cells on the media. Next day, select five different colonies, and amplify them in 30 μl of LB Borth (MDBio) containing 12.5 μg/ml chloramphenicol (Amresco) in a shaker at a temperature of 37° C. for 3 hours. Respectively take 1 μL of the cell liquids as templates. Undertake PCR of the cell liquids in RP11-29C11, using (SEQ ID No: 41; SEQ ID No: 42) as the primer. Undertake PCR of the cell liquids in CTD-32094, using (SEQ ID No: 43; SEQ ID No: 44) as the primer. Use a sample of pure water as the control group (BC). Examine the products of PCR with gel electrophoresis, and show the results in FIG. 6A. The qualified products of the PCR in RP11-29C11 should be greater than 140 bp. The qualified products of the PCR in CTD-32094 should be greater than 102 bp. Amplify the qualified colonies in 400 ml of LB Borth (MDBio) containing 12.5 μg/ml chloramphenicol (Amresco) in a shaker at a temperature of 37° C. for one night. Use the NucleoBond BAC 100 kit (Macherey-Nagel) to extract DNA of BAC clone RP11-29C11 and DNA of BAC clone CTD-32094. Measure the concentration and purity of DNA with the NanoDrop fluorescence absorption method, and show the results in FIG. 6B, wherein the single peaks of the absorption spectra indicate that none organic or protein impurity exists. Use nick translation to cut DNA of BAC clone RP11-29C11 and DNA of BAC clone CTD-32094 into fragments having a size of about 500 bp, as shown in FIG. 6C. Label RP11-29C11 with Biotin-11-2′-deoxyuridine-5′-triphosphate (Roche). Label CTD-32094 with digoxigenin-11-2′-deoxyuridine-5′-triphosphate (Roche). Thus is completed the preparation of fluorescent probes.

c. Hybridization of Sample Glass and Fluorescent Probe

In the hybridization process, use the human cot DNA (Invitrogen) and the salmon sperm DNA (Sigma) to isolate the non-specific repeated fragments. Hybridize the fragments in a humidified box at a temperature of 37° C. for one night. Cultivate the SAT cell line in a T45 culture box until the cells have occupied 80% of the volume. Add 0.2 ml of EtBr (1 mg/ml) to the cells, and place them still at a temperature of 37° C. for 90 minutes. Immunologically stain the fragments labeled by Biotin-11-T-deoxyuridine-5′-triphosphate with Biotinlated anti-avidin (Vector) and avidin-FITC (Vector) in sequence. Immunologically stain the fragments labeled by digoxigenin-11-2′-deoxyuridine-5′-triphosphate with sheep anti-digoxigenin and TRITC-conjugated F(ab′)2 fragment of rabbit anti-sheep. Undertake contrast staining of the stained fragments with DAPI, and observe the fragments with a microscope.

B. Experimental Results

FIG. 6D and FIG. 6E respectively show the cells without chromosome translocation and the cells with chromosome translocation. It is known from FIG. 6E that translocations occur in Chromosome 17q21.2 and Chromosome 12q13.13 of the OSCC SAT cell line.

### Embodiment III

### Popularization Rate of Gene Fusion in Cervical Squamous-Cell Cancer

A. Test Material and Test Method

The test material includes samples of cervical squamous-cell cancer (n=30), which are provided by the tissue bank of the China Medical University Hospital. The RNA of the samples is extracted with the RNeasy mini kit (Qiagen), quantified with the Nanodrop fluorescent absorption method, and analyzed with a gel-electrophoresis method. The high capacity cDNA RT kit (Applied Bioscience) is used to reverse-transcript 1 μg of RNA of each sample into cDNA. The cDNA is diluted by a 0.1×TE buffer solution to have a concentration of 50-80 ng/nl. Use 1 μL of cDNA as the template to undertake PCR with GAPDH gene sequence (SEQ ID No: 3; SEQ ID No: 4) being the primer. Examine the products of PCR with gel-electrophoresis, and discard the GAPDH-negative samples. Use 1 μL of cDNA taken from each APP-positive sample as the template to undertake PCR with the gene fusion sequence KRT6: KRT14 (SEQ ID No: 5; SEQ ID No: 6) being the external primer. Dilute the product of PCR with ten times of molecular-biological grade water. Use 1 μL of the diluted PCR product as the template to undertake nested-PCR with the gene fusion sequence KRT6: KRT14 (SEQ ID No: 7; SEQ ID No: 8) being the internal primer. Examine the product of the nested-PCR with gel electrophoresis.

B. Test Results

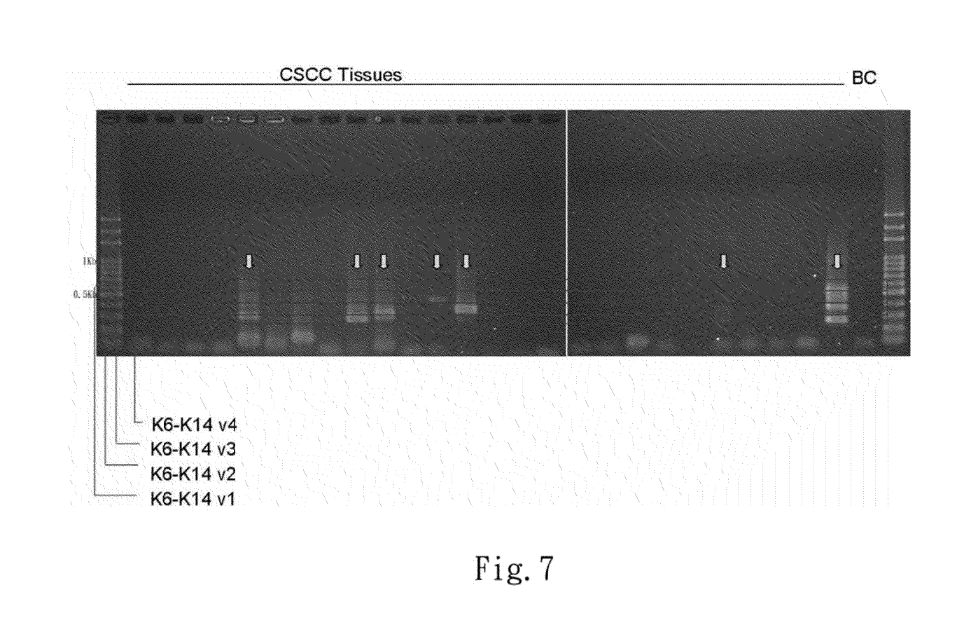

Prepare 32 samples of CSCC (Cervical Squamous-Cell Cancer) tissues and 1 sample of pure water (BC) as the templates. Undertake nested-PCR with the gene fusion sequence KRT6: KRT14 being the primer, and obtain the results shown in FIG. 7. There are 7 samples of CSCC tissues having positive reactions, which are indicated by the arrows in FIG. 7. The sample of pure water (BC) has negative reaction.

It is known from Embodiment I that the four groups of PCR products in gel electrophoresis all belong to the KRT6: KRT14 gene fusion sequences. Therefore, the popularization rate of the KRT6: KRT14 gene fusion is 26.9% (7/20) in the CSCC samples.

### Embodiment IV

### Popularization Rate of Gene Fusion in Nasopharyngeal Squamous-Cell Cancer

A. Test Material and Test Method

The test material includes samples of nasopharyngeal squamous-cell cancer (n=30), which are provided by the tissue bank of the China Medical University Hospital. The RNA of the samples is extracted with the RNeasy mini kit (Qiagen), quantified with the Nanodrop fluorescent absorption method, and analyzed with a gel-electrophoresis method. The high capacity cDNA RT kit (Applied Bioscience) is used to reverse-transcript 1 μg of RNA of each sample into cDNA. The cDNA is diluted by a 0.1×TE buffer solution to have a concentration of 50-80 ng/nl. Use 1 μL of cDNA as the template to undertake PCR with APP gene sequence (SEQ ID No: 1; SEQ ID No: 2) being the primer. Examine the products of PCR with gel-electrophoresis, and discard the APP-negative samples. Use 1 μL of cDNA taken from each APP-positive sample as the template to undertake PCR with the gene fusion sequence KRT6: KRT14 (SEQ ID No: 5; SEQ ID No: 6) being the external primer. Dilute the product of PCR with ten times of molecular-biological grade water. Use 1 μL of the diluted PCR product as the template to undertake nested-PCR with the gene fusion sequence KRT6: KRT14 (primer 132, SEQ ID No: 7; primer 216, SEQ ID No: 8) being the internal primer. Examine the product of the nested-PCR with gel electrophoresis.

B. Test Results

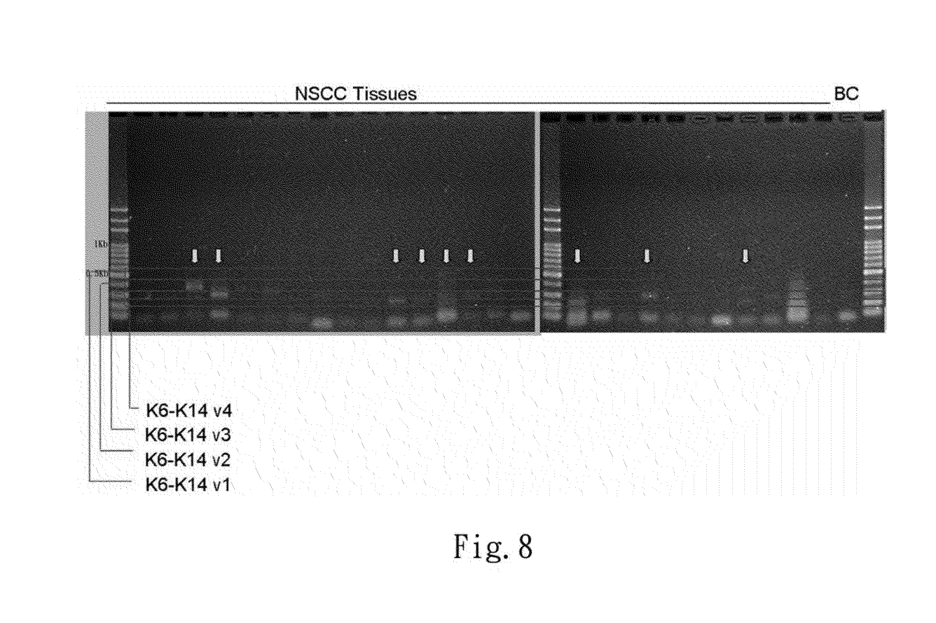

Prepare 27 samples of NSCC (Nasopharyngeal Squamous-Cell Cancer) tissues and 1 sample of pure water (BC) as the templates. Undertake nested-PCR with the gene fusion sequence KRT6: KRT14 being the primer, and obtain the results shown in FIG. 8. There are 9 samples of NSCC tissues having positive reactions, which are indicated by the arrows in FIG. 8. The sample of pure water (BC) has negative reaction. It is known from Embodiment I that the four groups of PCR products in gel electrophoresis all belong to the KRT6: KRT14 gene fusion sequences. Therefore, the popularization rate of the KRT6: KRT14 gene fusion is 33.39% (9/27) in the NSCC samples.

### Embodiment V

### Popularization Rate of Gene Fusion in Esophageal Squamous-Cell Cancer

A. Test material and test method

The test material includes samples of esophageal squamous-cell cancer (n=30), which are provided by the tissue bank of the China Medical University Hospital. The RNA of the samples is extracted with the RNeasy mini kit (Qiagen), quantified with the Nanodrop fluorescent absorption method, and analyzed with a gel-electrophoresis method. The high capacity cDNA RT kit (Applied Bioscience) is used to reverse-transcript 1 μg of RNA of each sample into cDNA. The cDNA is diluted by a 0.1×TE buffer solution to have a concentration of 50-80 ng/nl. Use of cDNA as the template to undertake PCR with APP gene sequence (SEQ ID No: 1; SEQ ID No: 2) being the primer. Examine the products of PCR with gel-electrophoresis, and discard the APP-negative samples. Use 1 μL of cDNA taken from each APP-positive sample as the template to undertake PCR with the gene fusion sequence KRT6: KRT14 (SEQ ID No: 5; SEQ ID No: 6) being the external primer. Dilute the product of PCR with ten times of molecular-biological grade water. Use 1 μL of the diluted PCR product as the template to undertake nested-PCR with the gene fusion sequence KRT6: KRT14 (primer 132, SEQ ID No: 7; primer 216, SEQ ID No: 8) being the internal primer. Examine the product of the nested-PCR with gel electrophoresis.

B. Test Results

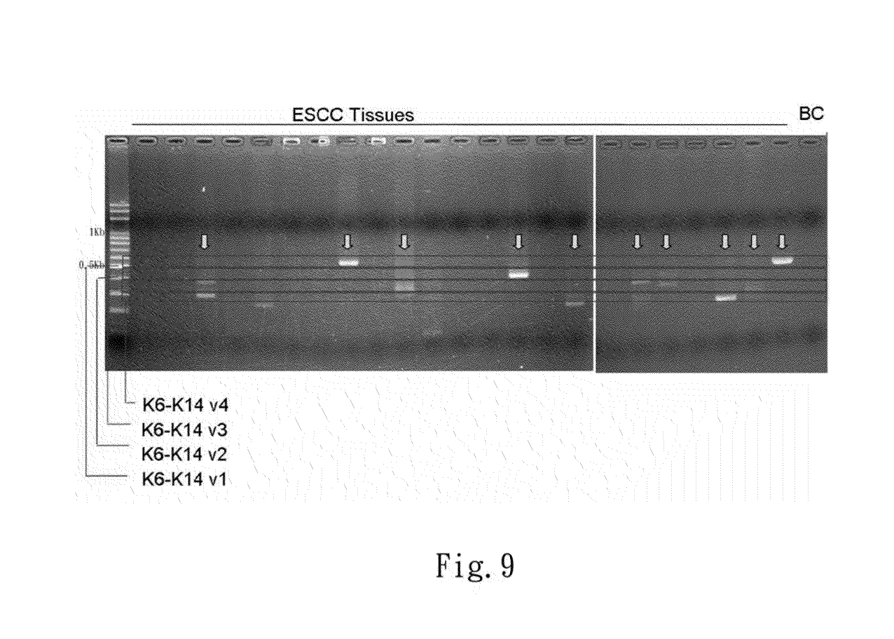

Prepare 23 samples of ESCC (Esophageal Squamous-Cell Cancer) tissues and 1 sample of pure water (BC) as the templates. Undertake nested-PCR with the gene fusion sequence KRT6: KRT14 being the primer, and obtain the results shown in FIG. 9. There are 10 samples of ESCC tissues having positive reactions, which are indicated by the arrows in FIG. 9. The sample of pure water (BC) has negative reaction. It is known from Embodiment I that the four groups of PCR products in gel electrophoresis all belong to the KRT6: KRT14 gene fusion sequences. Therefore, the popularization rate of the KRT6: KRT14 gene fusion is 43.5% (10/23) in the ESCC samples.

In conclusion, the present invention detects whether the squamous-cell sample of a testee has the chromosome translocation, mRNA transcript, protein of gene fusion, which is specific to the squamous-cell cancer and not expressed in healthy tissue. Therefore, the present invention is dedicated to examining squamous-cell cancer. The present invention has squamous-cell cancer specificity and would not be influenced by the surrounding healthy tissue.

The present invention possesses utility, novelty and non-obviousness and meets the condition for a patent. Thus, Inventors file the application for a patent. It is appreciated if the patent is approved fast.

The present invention has been described in detail with the embodiments. However, these embodiments are only to exemplify the present invention but not to limit the scope of the present invention. Any equivalent modification or variation according to the spirit of the present invention is to be also included within the scope of the present invention.

What is claimed is:

1. A method for in vitro detecting keratin gene fusion of squamous-cell cancer, comprising
Step (a): obtaining a sample of squamous cells; and
Step (b): detecting whether gene fusion occurs in the sample of squamous cells, wherein the gene fusion includes a 5′ terminal having a type I keratin gene, and a 3′ terminal having a type II keratin gene, a DSP gene, an MYH9 gene or an SFN gene;
wherein the sample of squamous cells is determined to have squamous-cell cancer if the gene fusion exists in the sample of squamous cells.

2. The method for detecting in vitro keratin gene fusion of squamous-cell cancer according to claim 1, wherein the type I keratin is selected from a group consisting of genes of KRT14, KRT16, KRT17, KRT18, KRT19, and KRT20; the type II keratin is selected from a group consisting of genes of KRT6A, KRT6B, KRT6C, KRT5, KRT7, and KRT8; the KRT14 gene has a nucleotide sequence SEQ ID No: 15; the KRT16 gene has a nucleotide sequence SEQ ID No: 16; the KRT17 gene has a nucleotide sequence SEQ ID No: 17; the KRT18 gene has a nucleotide sequence SEQ ID No: 35; the KRT19 gene has a nucleotide sequence SEQ ID No: 37; the KRT20 gene has a nucleotide sequence SEQ ID No: 39; the KRT6A gene has a nucleotide sequence SEQ ID No: 11; the KRT6B gene has a nucleotide sequence SEQ ID No: 12; the KRT6C gene has a nucleotide sequence SEQ ID No: 13; the KRT5 gene has a nucleotide sequence SEQ ID No: 14; the KRT7 gene has a nucleotide sequence SEQ ID No: 31; the KRT8 gene has a nucleotide sequence SEQ ID No: 33; the DSP gene has a nucleotide sequence SEQ ID No: 25; the MYH9 gene has a nucleotide sequence SEQ ID No: 26; the SFN gene has a nucleotide sequence SEQ ID No: 27.

3. The method for in vitro detecting keratin gene fusion of squamous-cell cancer according to claim 1, wherein Step (b) includes detecting whether the sample of squamous cells has chromosome translocation in genomic DNA, wherein the sequence of the chromosome translocation includes a 5′ terminal having a type I keratin gene, and a 3′ terminal having a type II keratin gene, a DSP gene, an MYH9 gene or an SFN gene.

4. The method for in vitro detecting keratin gene fusion of squamous-cell cancer according to claim 3, wherein the type I keratin is selected from a group consisting of genes of KRT14, KRT16, KRT17, KRT18, KRT19, and KRT20; the type II keratin is selected from a group consisting of genes of KRT6A, KRT6B, KRT6C, KRT5, KRT7, and KRT8; the KRT14 gene has a nucleotide sequence SEQ ID No: 15; the KRT16 gene has a nucleotide sequence SEQ ID No: 16; the KRT17 gene has a nucleotide sequence SEQ ID No: 17; the KRT18 gene has a nucleotide sequence SEQ ID No: 35; the KRT19 gene has a nucleotide sequence SEQ ID No: 37; the KRT20 gene has a nucleotide sequence SEQ ID No: 39; the KRT6A gene has a nucleotide sequence SEQ ID No: 11; the KRT6B gene has a nucleotide sequence SEQ ID No: 12; the KRT6C gene has a nucleotide sequence SEQ ID No: 13; the KRT5 gene has a nucleotide sequence SEQ ID No: 14; the KRT7 gene has a nucleotide sequence SEQ ID No: 31; the KRT8 gene has a nucleotide sequence SEQ ID No: 33; the DSP gene has a nucleotide sequence SEQ ID No: 25; the MYH9 gene has a nucleotide sequence SEQ ID No: 26; the SFN gene has a nucleotide sequence SEQ ID No: 27.

5. The method for in vitro detecting keratin gene fusion of squamous-cell cancer according to claim 1, wherein Step (b) includes detecting whether the sample of squamous cells has mRNA transcripts of gene fusion, wherein the mRNA transcript of gene fusion includes a 3′ terminal transcripted from a type I keratin gene, and a 5′ terminal transcripted from a type II keratin gene, a DSP gene, an MYH9 gene or an SFN gene.

6. The method for in vitro detecting keratin gene fusion of squamous-cell cancer according to claim 5, wherein the type I keratin is selected from a group consisting of genes of KRT14, KRT16, KRT17, KRT18, KRT19, and KRT20; the type II keratin is selected from a group consisting of genes of KRT6A, KRT6B, KRT6C, KRT5, KRT7, and KRT8; the KRT14 gene has a nucleotide sequence SEQ ID No: 15; the KRT16 gene has a nucleotide sequence SEQ ID No: 16; the KRT17 gene has a nucleotide sequence SEQ ID No: 17; the KRT18 gene has a nucleotide sequence SEQ ID No: 35; the KRT19 gene has a nucleotide sequence SEQ ID No: 37; the KRT20 gene has a nucleotide sequence SEQ ID No: 39; the KRT6A gene has a nucleotide sequence SEQ ID No: 11; the KRT6B gene has a nucleotide sequence SEQ ID No: 12; the KRT6C gene has a nucleotide sequence SEQ ID No: 13; the KRT5 gene has a nucleotide sequence SEQ ID No: 14; the KRT7 gene has a nucleotide sequence SEQ ID No: 31; the KRT8 gene has a nucleotide sequence SEQ ID No: 33; the DSP gene has a nucleotide sequence SEQ ID No: 25; the MYH9 gene has a nucleotide sequence SEQ ID No: 26; the SFN gene has a nucleotide sequence SEQ ID No: 27.

7. The method for in vitro detecting keratin gene fusion of squamous-cell cancer according to claim 1, wherein Step (b) includes detecting whether the sample of squamous cells has a gene fusion protein, wherein the gene fusion protein includes an N terminal having an amino acid sequence of a type I keratin, and a C terminal having an amino acid sequence of a type II keratin, a DSP protein, an MYH9 protein or an SFN protein.

8. The method for in vitro detecting keratin gene fusion of squamous-cell cancer according to claim 7, wherein the amino acid sequence of the type I keratin is selected from a group consisting of amino acid sequences of a KRT14 protein, a KRT16 protein, a KRT17 protein, a KRT18 protein, a KRT19 protein, and a KRT20 protein; the amino acid sequence of the type II keratin is selected from a group consisting of amino acid sequences of a KRT6A protein, a KRT6B protein, a KRT6C protein, a KRT5 protein, a KRT7 protein, and a KRT8 protein; the amino acid sequence of the KRT14 protein has an amino acid sequence SEQ ID No: 22; the amino acid sequence of the KRT16 protein has an amino acid sequence SEQ ID No: 23; the amino acid sequence of the KRT17 protein has an amino acid sequence SEQ ID No: 24; the amino acid sequence of the KRT18 protein has an amino acid sequence SEQ ID No: 36; the amino acid sequence of the KRT19 protein has an amino acid sequence SEQ ID No: 38; the amino acid sequence of the KRT20 protein has an amino acid sequence SEQ ID No: 40; the amino acid sequence of the KRT6A protein has an amino acid sequence SEQ ID No: 18; the amino acid sequence of the KRT6B protein has an amino acid sequence SEQ ID No: 19; the amino acid sequence of the KRT6C protein has an amino acid sequence SEQ ID No: 20; the amino acid sequence of the KRT5 protein has an amino acid sequence SEQ ID No: 21; the amino acid sequence of the KRT7 protein has an amino acid sequence SEQ ID No: 32; the amino acid sequence of the KRT8 protein has an amino acid sequence SEQ ID No: 34; the amino acid sequence of the DSP protein has an amino acid sequence SEQ ID No: 28; the amino acid sequence of the MYH9 protein has an amino acid sequence SEQ ID No: 29; the amino acid sequence of the SFN protein has an amino acid sequence SEQ ID No: 30.

9. The method for in vitro detecting keratin gene fusion of squamous-cell cancer according to claim 1, wherein the sample of squamous cells is selected from a group consisting of oral epithelial cells, cervical epithelial cells, nasopharyngeal epithelial cells and esophageal epithelial cells.

10. A method for in vitro detecting keratin gene fusion of squamous-cell cancer, comprising
Step (a): obtaining a sample of squamous cells; and
Step (b): detecting whether gene fusion occurs in the sample of squamous cells, wherein the gene fusion includes a 5′ terminal having a type II keratin gene, and a 3′ terminal having a type I keratin gene, a DSP gene, an MYH9 gene or an SFN gene;
wherein the sample of squamous cells is determined to have squamous-cell cancer if the gene fusion exists in the sample of squamous cells.

11. The method for in vitro detecting keratin gene fusion of squamous-cell cancer according to claim 10, wherein the type I keratin is selected from a group consisting of genes of KRT14, KRT16, KRT17, KRT18, KRT19, and KRT20; the type II keratin is selected from a group consisting of genes of KRT6A, KRT6B, KRT6C, KRT5, KRT7, and KRT8; the KRT14 gene has a nucleotide sequence SEQ ID No: 15; the KRT16 gene has a nucleotide sequence SEQ ID No: 16; the KRT17 gene has a nucleotide sequence SEQ ID No: 17; the KRT18 gene has a nucleotide sequence SEQ ID No: 35; the KRT19 gene has a nucleotide sequence SEQ ID No: 37; the KRT20 gene has a nucleotide sequence SEQ ID No: 39; the KRT6A gene has a nucleotide sequence SEQ ID No: 11; the KRT6B gene has a nucleotide sequence SEQ ID No: 12; the KRT6C gene has a nucleotide sequence SEQ ID No: 13; the KRT5 gene has a nucleotide sequence SEQ ID No: 14; the KRT7 gene has a nucleotide sequence SEQ ID No: 31; the KRT8 gene has a nucleotide sequence SEQ ID No: 33; the DSP gene has a nucleotide sequence SEQ ID No: 25; the MYH9 gene has a nucleotide sequence SEQ ID No: 26; the SFN gene has a nucleotide sequence SEQ ID No: 27.

12. The method for in vitro detecting keratin gene fusion of squamous-cell cancer according to claim 10, wherein Step (b) includes detecting whether the sample of squamous cells has chromosome translocation in genomic DNA, wherein the sequence of the chromosome translocation includes a 5′ terminal having a type II keratin gene, and a 3′ terminal having a type I keratin gene, a DSP gene, an MYH9 gene or an SFN gene.

13. The method for in vitro detecting keratin gene fusion of squamous-cell cancer according to claim 12, wherein the type I keratin is selected from a group consisting of genes of KRT14, KRT16, KRT17, KRT18, KRT19, and KRT20; the type II keratin is selected from a group consisting of genes of KRT6A, KRT6B, KRT6C, KRT5, KRT7, and KRT8; the KRT14 gene has a nucleotide sequence SEQ ID No: 15; the KRT16 gene has a nucleotide sequence SEQ ID No: 16; the KRT17 gene has a nucleotide sequence SEQ ID No: 17; the KRT18 gene has a nucleotide sequence SEQ ID No: 35; the KRT19 gene has a nucleotide sequence SEQ ID No: 37; the KRT20 gene has a nucleotide sequence SEQ ID No: 39; the KRT6A gene has a nucleotide sequence SEQ ID No: 11; the KRT6B gene has a nucleotide sequence SEQ ID No: 12; the KRT6C gene has a nucleotide sequence SEQ ID No: 13; the KRT5 gene has a nucleotide sequence SEQ ID No: 14; the KRT7 gene has a nucleotide sequence SEQ ID No: 31; the KRT8 gene has a nucleotide sequence SEQ ID No: 33; the DSP gene has a nucleotide sequence SEQ ID No: 25; the MYH9 gene has a nucleotide sequence SEQ ID No: 26; the SFN gene has a nucleotide sequence SEQ ID No: 27.

14. The method for in vitro detecting keratin gene fusion of squamous-cell cancer according to claim 10, wherein Step (b) includes detecting whether the sample of squamous cells has mRNA transcripts of gene fusion, wherein the mRNA transcript of gene fusion includes a 3′ terminal transcripted from a type II keratin gene, and a 5′ terminal transcripted from a type I keratin gene, a DSP gene, an MYH9 gene or an SFN gene.

15. The method for in vitro detecting keratin gene fusion of squamous-cell cancer according to claim 14, wherein the type I keratin is selected from a group consisting of genes of KRT14, KRT16, KRT17, KRT18, KRT19, and KRT20; the type II keratin is selected from a group consisting of genes of KRT6A, KRT6B, KRT6C, KRT5, KRT7, and KRT8; the KRT14 gene has a nucleotide sequence SEQ ID No: 15; the KRT16 gene has a nucleotide sequence SEQ ID No: 16; the KRT17 gene has a nucleotide sequence SEQ ID No: 17; the KRT18 gene has a nucleotide sequence SEQ ID No: 35; the KRT19 gene has a nucleotide sequence SEQ ID No: 37; the KRT20 gene has a nucleotide sequence SEQ ID No: 39; the KRT6A gene has a nucleotide sequence SEQ ID No: 11; the KRT6B gene has a nucleotide sequence SEQ ID No: 12; the KRT6C gene has a nucleotide sequence SEQ ID No: 13; the KRT5 gene has a nucleotide sequence SEQ ID No: 14; the KRT7 gene has a nucleotide sequence SEQ ID No: 31; the KRT8 gene has a nucleotide sequence SEQ ID No: 33; the DSP gene has a nucleotide sequence SEQ ID No: 25; the MYH9 gene has a nucleotide sequence SEQ ID No: 26; the SFN gene has a nucleotide sequence SEQ ID No: 27.

16. The method for in vitro detecting keratin gene fusion of squamous-cell cancer according to claim 10, wherein Step (b) includes detecting whether the sample of squamous cells has a gene fusion protein, wherein the gene fusion protein includes an N terminal having an amino acid sequence of a type II keratin, and a C terminal having an amino acid sequence of a type I keratin, a DSP protein, an MYH9 protein or an SFN protein.

17. The method for in vitro detecting keratin gene fusion of squamous-cell cancer according to claim 16, wherein the amino acid sequence of the type I keratin is selected from a group consisting of amino acid sequences of a KRT14 protein, a KRT16 protein, a KRT17 protein, a KRT18 protein, a KRT19 protein, and a KRT20 protein; the amino acid sequence of the type II keratin is selected from a group consisting of amino acid sequences of a KRT6A protein, a KRT6B protein, a KRT6C protein, a KRT5 protein, a KRT7 protein, and a KRT8 protein; the amino acid sequence of the KRT14 protein has an amino acid sequence SEQ ID No: 22; the amino acid sequence of the KRT16 protein has an amino acid sequence SEQ ID No: 23; the amino acid sequence of the KRT17 protein has an amino acid sequence SEQ ID No: 24; the amino acid sequence of the KRT18 protein has an amino acid sequence SEQ ID No: 36; the amino acid sequence of the KRT19 protein has an amino acid sequence SEQ ID No: 38; the amino acid sequence of the KRT20 protein has an amino acid sequence SEQ ID No: 40; the amino acid sequence of the KRT6A protein has an amino acid sequence SEQ ID No: 18; the amino acid sequence of the KRT6B protein has an amino acid sequence SEQ ID No: 19; the amino acid sequence of the KRT6C protein has an amino acid sequence SEQ ID No: 20; the amino acid sequence of the KRT5 protein has an amino acid sequence SEQ ID No: 21; the amino acid sequence of the KRT7 protein has an amino acid sequence SEQ ID No: 32; the amino acid sequence of the KRT8 protein has an amino acid sequence SEQ ID No: 34; the amino acid sequence of the DSP protein has an amino acid sequence SEQ ID No: 28; the amino acid sequence of the MYH9 protein has an amino acid sequence SEQ ID No: 29; the amino acid sequence of the SFN protein has an amino acid sequence SEQ ID No: 30.

18. The method for in vitro detecting keratin gene fusion of squamous-cell cancer according to claim 10, wherein the sample of squamous cells is selected from a group consisting of oral epithelial cells, cervical epithelial cells, nasopharyngeal epithelial cells and esophageal epithelial cells.

19. A method for in vitro detecting keratin gene fusion of squamous-cell cancer, comprising
Step (a): obtaining a sample of squamous cells; and
Step (b): detecting whether gene fusion occurs in the sample of squamous cells, wherein the gene fusion includes a 5′ terminal having a DSP gene, an MYH9 gene, or an SFN gene and a 3′ terminal having a type I keratin gene or a type II keratin gene;
wherein the sample of squamous cells is determined to have squamous-cell cancer if the gene fusion exists in the sample of squamous cells.

20. The method for in vitro detecting keratin gene fusion of squamous-cell cancer according to claim 19, wherein the type I keratin is selected from a group consisting of genes of KRT14, KRT16, KRT17, KRT18, KRT19, and KRT20; the type II keratin is selected from a group consisting of genes of KRT6A, KRT6B, KRT6C, KRT5, KRT7, and KRT8; the KRT14 gene has a nucleotide sequence SEQ ID No: 15; the KRT16 gene has a nucleotide sequence SEQ ID No: 16; the KRT17 gene has a nucleotide sequence SEQ ID No: 17; the KRT18 gene has a nucleotide sequence SEQ ID No: 35; the KRT19 gene has a nucleotide sequence SEQ ID No: 37; the KRT20 gene has a nucleotide sequence SEQ ID No: 39; the KRT6A gene has a nucleotide sequence SEQ ID No: 11; the KRT6B gene has a nucleotide sequence SEQ ID No: 12; the KRT6C gene has a nucleotide sequence SEQ ID No: 13; the KRT5 gene has a nucleotide sequence SEQ ID No: 14; the KRT7 gene has a nucleotide sequence SEQ ID No: 31; the KRT8 gene has a nucleotide sequence SEQ ID No: 33; the DSP gene has a nucleotide sequence SEQ ID No: 25; the MYH9 gene has a nucleotide sequence SEQ ID No: 26; the SFN gene has a nucleotide sequence SEQ ID No: 27.

21. The method for in vitro detecting keratin gene fusion of squamous-cell cancer according to claim 19, wherein Step (b) includes detecting whether the sample of squamous cells has chromosome translocation in genomic DNA, wherein the sequence of the genomic DNA includes a 5′ terminal having a DSP gene, an MYH9 gene or an SFN gene, and a 3′ terminal having a type I keratin gene or a type II keratin gene.

22. The method for in vitro detecting keratin gene fusion of squamous-cell cancer according to claim 21, wherein the type I keratin is selected from a group consisting of genes of KRT14, KRT16, KRT17, KRT18, KRT19, and KRT20; the type II keratin is selected from a group consisting of genes of KRT6A, KRT6B, KRT6C, KRT5, KRT7, and KRT8; the KRT14 gene has a nucleotide sequence SEQ ID No: 15; the KRT16 gene has a nucleotide sequence SEQ ID No: 16; the KRT17 gene has a nucleotide sequence SEQ ID No: 17; the KRT18 gene has a nucleotide sequence SEQ ID No: 35; the KRT19 gene has a nucleotide sequence SEQ ID No: 37; the KRT20 gene has a nucleotide sequence SEQ ID No: 39; the KRT6A gene has a nucleotide sequence SEQ ID No: 11; the KRT6B gene has a nucleotide sequence SEQ ID No: 12; the KRT6C gene has a nucleotide sequence SEQ ID No: 13; the KRT5 gene has a nucleotide sequence SEQ ID No: 14; the KRT7 gene has a nucleotide sequence SEQ ID No: 31; the KRT8 gene has a nucleotide sequence SEQ ID No: 33; the DSP gene has a nucleotide sequence SEQ ID No: 25; the MYH9 gene has a nucleotide sequence SEQ ID No: 26; the SFN gene has a nucleotide sequence SEQ ID No: 27.

23. The method for in vitro detecting keratin gene fusion of squamous-cell cancer according to claim 19, wherein Step (b) includes detecting whether the sample of squamous cells has mRNA transcript of gene fusion, wherein the mRNA transcript of gene fusion includes a 3′ terminal transcripted from a DSP gene, an MYH9 gene or an SFN gene, and a 5′ terminal transcripted from a type I keratin gene or a type II keratin gene.

24. The method for in vitro detecting keratin gene fusion of squamous-cell cancer according to claim 23, wherein the type I keratin is selected from a group consisting of genes of KRT14, KRT16, KRT17, KRT18, KRT19, and KRT20; the type II keratin is selected from a group consisting of genes of KRT6A, KRT6B, KRT6C, KRT5, KRT7, and KRT8; the KRT14 gene has a nucleotide sequence SEQ ID No: 15; the KRT16 gene has a nucleotide sequence SEQ ID No: 16; the KRT17 gene has a nucleotide sequence SEQ ID No: 17; the KRT18 gene has a nucleotide sequence SEQ ID No: 35; the KRT19 gene has a nucleotide sequence SEQ ID No: 37; the KRT20 gene has a nucleotide sequence SEQ ID No: 39; the KRT6A gene has a nucleotide sequence SEQ ID No: 11; the KRT6B gene has a nucleotide sequence SEQ ID No: 12; the KRT6C gene has a nucleotide sequence SEQ ID No: 13; the KRT5 gene has a nucleotide sequence SEQ ID No: 14; the KRT7 gene has a nucleotide sequence SEQ ID No: 31; the KRT8 gene has a nucleotide sequence SEQ ID No: 33; the DSP gene has a nucleotide sequence SEQ ID No: 25; the MYH9 gene has a nucleotide sequence SEQ ID No: 26; the SFN gene has a nucleotide sequence SEQ ID No: 27.

25. The method for in vitro detecting keratin gene fusion of squamous-cell cancer according to claim 19, wherein Step (b) includes detecting whether the sample of squamous cells has a gene fusion protein, wherein the gene fusion protein includes an N terminal having the amino acid sequence of a DSP protein, an MYH9 protein or an SFN protein, and a C terminal having the amino acid sequence of a type I keratin a type II keratin.

26. The method for in vitro detecting keratin gene fusion of squamous-cell cancer according to claim 25, wherein the amino acid sequence of the type I keratin is selected from a group consisting of amino acid sequences of a KRT14 protein, a KRT16 protein, a KRT17 protein, a KRT18 protein, a KRT19 protein, and a KRT20 protein; the amino acid sequence of the type II keratin is selected from a group consisting of amino acid sequences of a KRT6A protein, a KRT6B protein, a KRT6C protein, a KRT5 protein, a KRT7 protein, and a KRT8 protein; the amino acid sequence of the KRT14 protein has an amino acid sequence SEQ ID No: 22; the amino acid sequence of the KRT16 protein has an amino acid sequence SEQ ID No: 23; the amino acid sequence of the KRT17 protein has an amino acid sequence SEQ ID No: 24; the amino acid sequence of the KRT18 protein has an amino acid sequence SEQ ID No: 36; the amino acid sequence of the KRT19 protein has an amino acid sequence SEQ ID No: 38; the amino acid sequence of the KRT20 protein has an amino acid sequence SEQ ID No: 40; the amino acid sequence of the KRT6A protein has an amino acid sequence SEQ ID No: 18; the amino acid sequence of the KRT6B protein has an amino acid sequence SEQ ID No: 19; the amino acid sequence of the KRT6C protein has an amino acid sequence SEQ ID No: 20; the amino acid sequence of the KRT5 protein has an amino acid sequence SEQ ID No: 21; the amino acid sequence of the KRT7 protein has an amino acid sequence SEQ ID No: 32; the amino acid sequence of the KRT8 protein has an amino acid sequence SEQ ID No: 34; the amino acid sequence of the DSP protein has an amino acid sequence SEQ ID No: 28; the amino acid sequence of the MYH9 protein has an amino acid sequence SEQ ID No: 29; the amino acid sequence of the SFN protein has an amino acid sequence SEQ ID No: 30.

27. The method for in vitro detecting keratin gene fusion of squamous-cell cancer according to claim 19, wherein the sample of squamous cells is selected from a group consisting of oral epithelial cells, cervical epithelial cells, nasopharyngeal epithelial cells and esophageal epithelial cells.


SEQUENCE LISTING (44 sequences)

SEQ ID NO: 1 - DNA, 23 bp, Homo sapiens
gtgaagatggatgcagaattccg

SEQ ID NO: 2 - DNA, 25 bp, Homo sapiens
aaagaacttgtaggttggattttcg

SEQ ID NO: 3 - DNA, 22 bp, Homo sapiens
ccatgccatcactgccacccag

SEQ ID NO: 4 - DNA, 22 bp, Homo sapiens
aggtccaccaccctgttgctgt

SEQ ID NO: 5 - DNA, 22 bp, Homo sapiens
cctcaaggatgccaagaacaag

SEQ ID NO: 6 - DNA, 22 bp, Homo sapiens
acgcaccttgtccaggtaggag

SEQ ID NO: 7 - DNA, 24 bp, Homo sapiens
gagtaccaggagctgatgaatgtc

SEQ ID NO: 8 - DNA, 22 bp, Homo sapiens
cttctcactgcccaccagaagc

SEQ ID NO: 9 - DNA, 2313 bp, Homo sapiens
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccatgtcccaaagtttactcaagactacccaggaacctcttgcccgagatcctgttaaacttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 10 - DNA, 1008 bp, Homo sapiens
atggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaa

SEQ ID NO: 11 - DNA, 1695 bp, Homo sapiens
atggccagcacatccaccaccatcaggagccacagcagcagccgccggggtttcagtgccaactcagccaggctccctggggtcagccgctctggcttcagcagcgtctccgtgtcccgctccaggggcagtggtggcctgggtggtgcatgtggaggagctggctttggcagccgcagtctgtatggcctggggggctccaagaggatctccattggagggggcagctgtgccatcagtggcggctatggcagcagagccggaggcagctatggctttggtggcgccgggagtggatttggtttcggtggtggagccggcattggctttggtctgggtggtggagccggccttgctggtggctttgggggccctggcttccctgtgtgcccccctggaggcatccaagaggtcaccgtcaaccagagtctcctgactcccctcaacctgcaaatcgatcccaccatccagcgggtgcgggctgaggagcgtgaacagatcaagaccctcaacaacaagtttgcctccttcatcgacaaggtgcggttcctggagcagcagaacaaggttctggaaacaaagtggaccctgctgcaggagcagggcaccaagactgtgaggcagaacctggagccgttgttcgagcagtacatcaacaacctcaggaggcagctggacagcattgtcggggaacggggccgcctggactcagagctcagaggcatgcaggacctggtggaggacttcaagaacaaatatgaggatgaaatcaacaagcgcacagcagcagagaatgaatttgtgactctgaagaaggatgtggatgctgcctacatgaacaaggttgaactgcaagccaaggcagacactctcacagacgagatcaacttcctgagagccttgtatgatgcagagctgtcccagatgcagacccacatctcagacacatctgtggtgctgtccatggacaacaaccgcaacctggacctggacagcatcatcgctgaggtcaaggcccaatatgaggagattgctcagagaagccgggctgaggctgagtcctggtaccagaccaagtacgaggagctgcaggtcacagcaggcagacatggggacgacctgcgcaacaccaagcaggagattgctgagatcaaccgcatgatccagaggctgagatctgagatcgaccacgtcaagaagcagtgcgccaacctgcaggccgccattgctgatgctgagcagcgtggggagatggccctcaaggatgccaagaacaagctggaagggctggaggatgccctgcagaaggccaagcaggacctggcccggctgctgaaggagtaccaggagctgatgaatgtcaagctggccctggacgtggagatcgccacctaccgcaagctgctggagggtgaggagtgcaggctgaatggcgaaggcgttggacaagtcaacatctctgtggtgcagtccaccgtctccagtggctatggcggtgccagtggtgtcggcagtggcttaggcctgggtggaggaagcagctactcctatggcagtggtcttggcgttggaggtggcttcagttccagcagtggcagagccattgggggtggcctcagctctgttggaggcggcagttccaccatcaagtacaccaccacctcctcctccagcaggaagagctataagcactaa

SEQ ID NO: 12 - DNA, 1695 bp, Homo sapiens
atggccagcacatccaccaccatcaggagccacagcagcagccgccggggtttcagtgccaactcagccaggctccctggggtcagccgctctggcttcagcagcatctccgtgtcccgctccaggggcagtggtggcctgggtggcgcatgtggaggagctggctttggcagccgcagtctgtatggcctggggggctccaagaggatctccattggagggggcagctgtgccatcagtggcggctatggcagcagagccggaggcagctatggctttggtggcgccgggagtggatttggtttcggtggtggagccggcattggctttggtctgggtggtggagccggccttgctggtggctttgggggccctggcttccctgtgtgcccccctggaggcatccaagaggtcactgtcaaccagagtctcctgactcccctcaacctgcaaattgaccccgccatccagcgggtgcgggccgaggagcgtgagcagatcaagaccctcaacaacaagtttgcctccttcatcgacaaggtgcggttcctagagcagcagaacaaggttctggacaccaagtggaccctgctgcaggagcagggcaccaagactgtgaggcagaacctggagccgttgttcgagcagtacatcaacaacctcaggaggcagctggacaacatcgtgggggaacggggtcgtctggactcggagctgagaaacatgcaggacctggtggaggacctcaagaacaaatatgaggatgaaatcaacaagcgcacagcagcagagaatgaatttgtgactctgaagaaggatgtggatgctgcctacatgaacaaggttgaactgcaagccaaggcagacactcttacagatgagatcaacttcctgagagccttgtatgatgcagagctgtcccagatgcagacccacatctcagacacatccgtggtgctatccatggacaacaaccgcaacctggacctggacagcatcatcgctgaggtcaaggcccaatatgaggagattgctcagaggagcagggctgaggctgagtcctggtaccagacaaagtacgaggagctgcagatcacagcaggcagacatggggacgacctgcgcaacaccaagcaggagattgctgagatcaaccgcatgatccagaggctgagatctgagatcgaccacgtcaagaagcagtgtgccaacctacaggccgccattgctgatgctgagcagcgtggggagatggccctcaaggatgctaagaacaagctggaagggctggaggatgccctgcagaaggccaagcaggacctggcccggctgctgaaggagtaccaggagctgatgaacgtcaagctggccctggatgtggagatcgccacctaccgcaagctgctggagggcgaggagtgcaggctgaatggcgaaggcgttggacaagtcaacatctctgtagtgcagtccaccgtctccagtggctatggcggtgccagcggtgtcggcagtggcttaggcctgggtggaggaagcagctactcctatggcagtggtcttggcgttggaggcggctttagttccagcagcggcagagccactgggggtggcctcagctctgttggaggcggcagttccaccatcaagtacaccaccacctcctcctccagcaggaagagctacaagcactga

SEQ ID NO: 13 - DNA, 1695 bp, Homo sapiens
atggccagcacatccaccaccatcaggagccacagcagcagccgccggggtttcagtgccaactcagccaggctccctggggtcagccgctctggcttcagcagcatctccgtgtcccgctccaggggcagtggtggcctgggtggtgcatgtggaggagctggctttggcagccgcagtctgtatggcctggggggctccaagaggatctccattggagggggcagctgtgccatcagtggcggctatggcagcagagccggaggcagctatggctttggtggcgccgggagtggatttggtttcggtggtggagccggcattggctttggtctgggtggtggagccggccttgctggtggctttgggggccctggcttccctgtgtgcccccctggaggcatccaagaggtcaccgtcaaccagagtctcctgactcccctcaacctgcaaattgaccccgccatccagcgggtgcgggccgaggagcgtgagcagatcaagaccctcaacaacaagtttgcctccttcatcgacaaggtgcggttcctagagcagcagaacaaggttctggacaccaagtggaccctgctgcaggagcagggcaccaagactgtgaggcagaacctggagccgttgttcgagcagtacatcaacaacctcaggaggcagctggacagcatcgtcggggaacggggccgcctggactcggagctgagaaacatgcaggacctggtggaggacctcaagaacaaatatgaggatgaaatcaacaagcgcacagcagcagagaatgaatttgtgactctgaagaaggatgtggatgctgcctacatgaacaaggttgaactgcaagccaaggcagacactctcacagatgagatcaacttcctgagagccttgtatgatgcagagctgtcccagatgcagacccacatctcagacacatccgtggtgctatccatggacaacaaccgcaacctggacctggacagcatcatcgctgaggtcaaggcccaatacgaggagattgctcagaggagccgggctgaggctgagtcctggtaccagaccaagtacgaggagctgcaggtcacagcaggcagacatggggacgacctgcgcaacaccaagcaggagattgctgagatcaaccgcatgatccagaggctgagatctgagatcgaccatgtcaagaagcagtgtgccagcctgcaggctgccattgctgatgctgagcagcgtggggagatggcactcaaggatgctaagaacaagctggaagggctggaggatgccctgcagaaggccaagcaggacctggcccggctgctgaaggagtaccaggagctgatgaatgtcaagctggccctggatgtggagatcgccacctaccgcaagctgctggagggcgaggagtgcaggctgaatggcgaaggcgttggacaagtcaacgtctctgtagtacagtccaccatctccagtggctatggcggtgccagcggtgtcggcagtggcttaggcctgggtggaggaagcagctactcctatggcagtggtcttggcattggaggtggcttcagttccagcagtggcagagccattgggggtggcctcagctctgttggaggcggcagttccaccatcaagtacaccaccacctcctcctccagcaggaagagctacaagcactaa

SEQ ID NO: 14 - DNA, 1773 bp, Homo sapiens
atgtctcgccagtcaagtgtgtccttccggagcgggggcagtcgtagcttcagcaccgcctctgccatcaccccgtctgtctcccgcaccagcttcacctccgtgtcccggtccgggggtggcggtggtggtggcttcggcagggtcagccttgcgggtgcttgtggagtgggtggctatggcagccggagcctctacaacctggggggctccaagaggatatccatcagcactagtggtggcagcttcaggaaccggtttggtgctggtgctggaggcggctatggctttggaggtggtgccggtagtggatttggtttcggcggtggagctggtggtggctttgggctcggtggcggagctggctttggaggtggcttcggtggccctggctttcctgtctgccctcctggaggtatccaagaggtcactgtcaaccagagtctcctgactcccctcaacctgcaaatcgaccccagcatccagagggtgaggaccgaggagcgcgagcagatcaagaccctcaacaataagtttgcctccttcatcgacaaggtgcggttcctggagcagcagaacaaggttctggacaccaagtggaccctgctgcaggagcagggcaccaagactgtgaggcagaacctggagccgttgttcgagcagtacatcaacaacctcaggaggcagctggacagcatcgtgggggaacggggccgcctggactcagagctgagaaacatgcaggacctggtggaagacttcaagaacaagtatgaggatgaaatcaacaagcgtaccactgctgagaatgagtttgtgatgctgaagaaggatgtagatgctgcctacatgaacaaggtggagctggaggccaaggttgatgcactgatggatgagattaacttcatgaagatgttctttgatgcggagctgtcccagatgcagacgcatgtctctgacacctcagtggtcctctccatggacaacaaccgcaacctggacctggatagcatcatcgctgaggtcaaggcccagtatgaggagattgccaaccgcagccggacagaagccgagtcctggtatcagaccaagtatgaggagctgcagcagacagctggccggcatggcgatgacctccgcaacaccaagcatgagatctctgagatgaaccggatgatccagaggctgagagccgagattgacaatgtcaagaaacagtgcgccaatctgcagaacgccattgcggatgccgagcagcgtggggagctggccctcaaggatgccaggaacaagctggccgagctggaggaggccctgcagaaggccaagcaggacatggcccggctgctgcgtgagtaccaggagctcatgaacaccaagctggccctggacgtggagatcgccacttaccgcaagctgctggagggcgaggaatgcagactcagtggagaaggagttggaccagtcaacatctctgttgtcacaagcagtgtttcctctggatatggcagtggcagtggctatggcggtggcctcggtggaggtcttggcggcggcctcggtggaggtcttgccggaggtagcagtggaagctactactccagcagcagtgggggtgtcggcctaggtggtgggctcagtgtggggggctctggcttcagtgcaagcagtggccgagggctgggggtgggctttggcagtggcgggggtagcagctccagcgtcaaatttgtctccaccacctcctcctcccggaagagcttcaagagctaa

SEQ ID NO: 15 - DNA, 1419 bp, Homo sapiens
atgaccacctgcagccgccagttcacctcctccagctccatgaagggctcctgcggcatcgggggcggcatcgggggcggctccagccgcatctcctccgtcctggccggagggtcctgccgcgcccccagcacctacgggggcggcctgtctgtctcatcctcccgcttctcctctgggggagcctgcgggctggggggcggctatggcggtggcttcagcagcagcagcagcagctttggtagtggctttgggggaggatatggtggtggccttggtgctggcttgggtggtggctttggtggtggctttgctggtggtgatgggcttctggtgggcagtgagaaggtgaccatgcagaacctcaatgaccgcctggcctcctacctggacaaggtgcgtgctctggaggaggccaacgccgacctggaagtgaagatccgtgactggtaccagaggcagcggcctgctgagatcaaagactacagtccctacttcaagaccattgaggacctgaggaacaagattctcacagccacagtggacaatgccaatgtccttctgcagattgacaatgcccgtctggccgcggatgacttccgcaccaagtatgagacagagttgaacctgcgcatgagtgtggaagccgacatcaatggcctgcgcagggtgctggacgaactgaccctggccagagctgacctggagatgcagattgagagcctgaaggaggagctggcctacctgaagaagaaccacgaggaggagatgaatgccctgagaggccaggtgggtggagatgtcaatgtggagatggacgctgcacctggcgtggacctgagccgcattctgaacgagatgcgtgaccagtatgagaagatggcagagaagaaccgcaaggatgccgaggaatggttcttcaccaagacagaggagctgaaccgcgaggtggccaccaacagcgagctggtgcagagcggcaagagcgagatctcggagctccggcgcaccatgcagaacctggagattgagctgcagtcccagctcagcatgaaagcatccctggagaacagcctggaggagaccaaaggtcgctactgcatgcagctggcccagatccaggagatgattggcagcgtggaggagcagctggcccagctccgctgcgagatggagcagcagaaccaggagtacaagatcctgctggacgtgaagacgcggctggagcaggagatcgccacctaccgccgcctgctggagggcgaggacgcccacctctcctcctcccagttctcctctggatcgcagtcatccagagatgtgacctcctccagccgccaaatccgcaccaaggtcatggatgtgcacgatggcaaggtggtgtccacccacgagcaggtccttcgcaccaagaactga

SEQ ID NO: 16 - DNA, 1422 bp, Homo sapiens
atgaccacctgcagccgccagttcacctcctccagctccatgaagggctcctgcggcatcggaggcggcatcgggggcggctccagccgcatctcctccgtcctggccggagggtcctgccgtgcccccagcacctacgggggcggcctgtctgtctcctctcgcttctcctctgggggagcctgcgggctggggggcggctatggcggtggcttcagcagcagcagcagctttggtagtggcttcgggggaggatatggtggtggccttggtgctggcttcggtggtggcttgggtgctggctttggtggtggttttgctggtggtgatgggcttctggtgggcagtgagaaggtgaccatgcagaacctcaatgaccgcctggcctcctacctggacaaggtgcgtgctctggaggaggccaacgccgacctggaagtgaagatccgtgactggtaccagaggcagcggcccagtgagatcaaagactacagtccctacttcaagaccatcgaggacctgaggaacaagatcattgcggccaccattgagaatgcgcagcccattttgcagattgacaatgccaggctggcagccgatgacttcaggaccaagtatgagcatgaactggccctgcggcagactgtggaggccgacgtcaatggcctgcgccgggtgttggatgagctgaccctggccaggactgacctggagatgcagatcgaaggcctgaaggaggagctggcctacctgaggaagaaccacgaggaggagatgcttgctctgagaggtcagaccggcggagatgtgaacgtggagatggatgctgcacctggcgtggacctgagccgcatcctgaatgagatgcgtgaccagtacgagcagatggcagagaaaaaccgcagagacgctgagacctggttcctgagcaagaccgaggagctgaacaaagaagtggcctccaacagcgaactggtacagagcagccgcagtgaggtgacggagctccggagggtgctccagggcctggagattgagctgcagtcccagctcagcatgaaagcatccctggagaacagcctggaggagaccaaaggccgctactgcatgcagctgtcccagatccagggactgattggcagtgtggaggagcagctggcccagctacgctgtgagatggagcagcagagccaggagtaccagatcttgctggatgtgaagacgcggctggagcaggagattgccacctaccgccgcctgctggagggcgaggatgcccacctttcctcccagcaagcatctggccaatcctattcttcccgcgaggtcttcacctcctcctcgtcctcttcgagccgtcagacccggcccatcctcaaggagcagagctcatccagcttcagccagggccagagctcctag

SEQ ID NO: 17 - DNA, 1299 bp, Homo sapiens
atgaccacctccatccgccagttcacctcctccagctccatcaagggctcctccggcctggggggcggctcgtcccgcacctcctgccggctgtctggcggcctgggtgccggctcctgcaggctgggatctgctggcggcctgggcagcaccctcgggggtagcagctactccagctgctacagctttggctctggtggtggctatggcagcagctttgggggtgttgatgggctgctggctggaggtgagaaggccaccatgcagaacctcaatgaccgcctggcctcctacctggacaaggtgcgtgccctggaggaggccaacactgagctggaggtgaagatccgtgactggtaccagaggcaggccccggggcccgcccgtgactacagccagtactacaggacaattgaggagctgcagaacaagatcctcacagccaccgtggacaatgccaacatcctgctacagattgacaatgcccgtctggctgctgatgacttccgcaccaagtttgagacagagcaggccctgcgcctgagtgtggaggccgacatcaatggcctgcgcagggtgctggatgagctgaccctggccagagccgacctggagatgcagattgagaacctcaaggaggagctggcctacctgaagaagaaccacgaggaggagatgaacgccctgcgaggccaggtgggtggtgagatcaatgtggagatggacgctgccccaggcgtggacctgagccgcatcctcaacgagatgcgtgaccagtatgagaagatggcagagaagaaccgcaaggatgccgaggattggttcttcagcaagacagaggaactgaaccgcgaggtggccaccaacagtgagctggtgcagagtggcaagagtgagatctcggagctccggcgcaccatgcaggccttggagatagagctgcagtcccagctcagcatgaaagcatccctggagggcaacctggcggagacagagaaccgctactgcgtgcagctgtcccagatccaggggctgattggcagcgtggaggagcagctggcccagcttcgctgcgagatggagcagcagaaccaggaatacaaaatcctgctggatgtgaagacgcggctggagcaggagattgccacctaccgccgcctgctggagggagaggatgcccacctgactcagtacaagaaagaaccggtgaccacccgtcaggtgcgtaccattgtggaagaggtccaggatggcaaggtcatctcctcccgcgagcaggtccaccagaccacccgctga

SEQ ID NO: 18 - protein, 564 aa, Homo sapiens
MASTSTTIRSHSSSRRGFSANSARLPGVSRSGFSSVSVSRSRGSGGLGGACGGAGFGSRSLYGLGGSKRISIGGGSCAISGGYGSRAGGSYGFGGAGSGFGFGGGAGIGFGLGGGAGLAGGFGGPGFPVCPPGGIQEVTVNQSLLTPLNLQIDPTIQRVRAEEREQIKTLNNKFASFIDKVRFLEQQNKVLETKWTLLQEQGTKTVRQNLEPLFEQYINNLRRQLDSIVGERGRLDSELRGMQDLVEDFKNKYEDEINKRTAAENEFVTLKKDVDAAYMNKVELQAKADTLTDEINFLRALYDAELSQMQTHISDTSVVLSMDNNRNLDLDSIIAEVKAQYEEIAQRSRAEAESWYQTKYEELQVTAGRHGDDLRNTKQEIAEINRMIQRLRSEIDHVKKQCANLQAAIADAEQRGEMALKDAKNKLEGLEDALQKAKQDLARLLKEYQELMNVKLALDVEIATYRKLLEGEECRLNGEGVGQVNISVVQSTVSSGYGGASGVGSGLGLGGGSSYSYGSGLGVGGGFSSSSGRAIGGGLSSVGGGSSTIKYTTTSSSSRKSYKH

SEQ ID NO: 19 - protein, 564 aa, Homo sapiens
MASTSTTIRSHSSSRRGFSANSARLPGVSRSGFSSISVSRSRGSGGLGGACGGAGFGSRSLYGLGGSKRISIGGGSCAISGGYGSRAGGSYGFGGAGSGFGFGGGAGIGFGLGGGAGLAGGFGGPGFPVCPPGGIQEVTVNQSLLTPLNLQIDPAIQRVRAEEREQIKTLNNKFASFIDKVRFLEQQNKVLDTKWTLLQEQGTKTVRQNLEPLFEQYINNLRRQLDNIVGERGRLDSELRNMQDLVEDLKNKYEDEINKRTAAENEFVTLKKDVDAAYMNKVELQAKADTLTDEINFLRALYDAELSQMQTHISDTSVVLSMDNNRNLDLDSIIAEVKAQYEEIAQRSRAEAESWYQTKYEELQITAGRHGDDLRNTKQEIAEINRMIQRLRSEIDHVKKQCANLQAAIADAEQRGEMALKDAKNKLEGLEDALQKAKQDLARLLKEYQELMNVKLALDVEIATYRKLLEGEECRLNGEGVGQVNISVVQSTVSSGYGGASGVGSGLGLGGGSSYSYGSGLGVGGGFSSSSGRATGGGLSSVGGGSSTIKYTTTSSSSRKSYKH

SEQ ID NO: 20 - protein, 564 aa, Homo sapiens
MASTSTTIRSHSSSRRGFSANSARLPGVSRSGFSSISVSRSRGSGGLGGACGGAGFGSRSLYGLGGSKRISIGGGSCAISGGYGSRAGGSYGFGGAGSGFGFGGGAGIGFGLGGGAGLAGGFGGPGFPVCPPGGIQEVTVNQSLLTPLNLQIDPAIQRVRAEEREQIKTLNNKFASFIDKVRFLEQQNKVLDTKWTLLQEQGTKTVRQNLEPLFEQYINNLRRQLDSIVGERGRLDSELRNMQDLVEDLKNKYEDEINKRTAAENEFVTLKKDVDAAYMNKVELQAKADTLTDEINFLRALYDAELSQMQTHISDTSVVLSMDNNRNLDLDSIIAEVKAQYEEIAQRSRAEAESWYQTKYEELQVTAGRHGDDLRNTKQEIAEINRMIQRLRSEIDHVKKQCASLQAAIADAEQRGEMALKDAKNKLEGLEDALQKAKQDLARLLKEYQELMNVKLALDVEIATYRKLLEGEECRLNGEGVGQVNVSVVQSTISSGYGGASGVGSGLGLGGGSSYSYGSGLGIGGGFSSSSGRAIGGGLSSVGGGSSTIKYTTTSSSSRKSYKH

SEQ ID NO: 21 - protein, 590 aa, Homo sapiens
MSRQSSVSFRSGGSRSFSTASAITPSVSRTSFTSVSRSGGGGGGGFGRVSLAGACGVGGYGSRSLYNLGGSKRISISTSGGSFRNRFGAGAGGGYGFGGGAGSGFGFGGGAGGGFGLGGGAGFGGGFGGPGFPVCPPGGIQEVTVNQSLLTPLNLQIDPSIQRVRTEEREQIKTLNNKFASFIDKVRFLEQQNKVLDTKWTLLQEQGTKTVRQNLEPLFEQYINNLRRQLDSIVGERGRLDSELRNMQDLVEDFKNKYEDEINKRTTAENEFVMLKKDVDAAYMNKVELEAKVDALMDEINFMKMFFDAELSQMQTHVSDTSVVLSMDNNRNLDLDSIIAEVKAQYEEIANRSRTEAESWYQTKYEELQQTAGRHGDDLRNTKHEISEMNRMIQRLRAEIDNVKKQCANLQNAIADAEQRGELALKDARNKLAELEEALQKAKQDMARLLREYQELMNTKLALDVEIATYRKLLEGEECRLSGEGVGPVNISVVTSSVSSGYGSGSGYGGGLGGGLGGGLGGGLAGGSSGSYYSSSSGGVGLGGGLSVGGSGFSASSGRGLGVGFGSGGGSSSSVKFVSTTSSSRKSFKS

SEQ ID NO: 22 - protein, 472 aa, Homo sapiens
MTTCSRQFTSSSSMKGSCGIGGGIGGGSSRISSVLAGGSCRAPSTYGGGLSVSSSRFSSGGAYGLGGGYGGGFSSSSSSFGSGFGGGYGGGLGAGLGGGFGGGFAGGDGLLVGSEKVTMQNLNDRLASYLDKVRALEEANADLEVKIRDWYQRQRPAEIKDYSPYFKTIEDLRNKILTATVDNANVLLQIDNARLAADDFRTKYETELNLRMSVEADINGLRRVLDELTLARADLEMQIESLKEELAYLKKNHEEEMNALRGQVGGDVNVEMDAAPGVDLSRILNEMRDQYEKMAEKNRKDAEEWFFTKTEELNREVATNSELVQSGKSEISELRRTMQNLEIELQSQLSMKASLENSLEETKGRYCMQLAQIQEMIGSVEEQLAQLRCEMEQQNQEYKILLDVKTRLEQEIATYRRLLEGEDAHLSSSQFSSGSQSSRDVTSSSRQIRTKVMDVHDGKVVSTHEQVLRTKN

SEQ ID NO: 23 - protein, 473 aa, Homo sapiens
MTTCSRQFTSSSSMKGSCGIGGGIGGGSSRISSVLAGGSCRAPSTYGGGLSVSSRFSSGGACGLGGGYGGGFSSSSSFGSGFGGGYGGGLGAGFGGGLGAGFGGGFAGGDGLLVGSEKVTMQNLNDRLASYLDKVRALEEANADLEVKIRDWYQRQRPSEIKDYSPYFKTIEDLRNKIIAATIENAQPILQIDNARLAADDFRTKYEHELALRQTVEADVNGLRRVLDELTLARTDLEMQIEGLKEELAYLRKNHEEEMLALRGQTGGDVNVEMDAAPGVDLSRILNEMRDQYEQMAEKNRRDAETWFLSKTEELNKEVASNSELVQSSRSEVTELRRVLQGLEIELQSQLSMKASLENSLEETKGRYCMQLSQIQGLIGSVEEQLAQLRCEMEQQSQEYQILLDVKTRLEQEIATYRRLLEGEDAHLSSQQASGQSYSSREVFTSSSSSSSRQTRPILKEQSSSSFSQGQSS

SEQ ID NO: 24 - protein, 432 aa, Homo sapiens
MTTSIRQFTSSSSIKGSSGLGGGSSRTSCRLSGGLGAGSCRLGSAGGLGSTLGGSSYSSCYSFGSGGGYGSSFGGVDGLLAGGEKATMQNLNDRLASYLDKVRALEEANTELEVKIRDWYQRQAPGPARDYSQYYRTIEELQNKILTATVDNANILLQIDNARLAADDFRTKFETEQALRLSVEADINGLRRVLDELTLARADLEMQIENLKEELAYLKKNHEEEMNALRGQVGGEINVEMDAAPGVDLSRILNEMRDQYEKMAEKNRKDAEDWFFSKTEELNREVATNSELVQSGKSEISELRRTMQALEIELQSQLSMKASLEGNLAETENRYCVQLSQIQGLIGSVEEQLAQLRCEMEQQNQEYKILLDVKTRLEQEIATYRRLLEGEDAHLTQYKKEPVTTRQVRTIVEEVQDGKVISSREQVHQTTR

SEQ ID NO: 25 - DNA, 8616 bp, Homo sapiens
atgagctgcaacggaggctcccacccgcggatcaacactctgggccgcatgatccgcgccgagtctggcccggacctgcgctacgaggtgaccagcggcggcgggggcaccagcaggatgtactattctcggcgcggcgtgatcaccgaccagaactcggacggctactgtcaaaccggcacgatgtccaggcaccagaaccagaacaccatccaggagctgctgcagaactgctccgactgcttgatgcgagcagagctcatcgtgcagcctgaattgaagtatggagatggaatacaactgactcggagtcgagaattggatgagtgttttgcccaggccaatgaccaaatggaaatcctcgacagcttgatcagagagatgcggcagatgggccagccctgtgatgcttaccagaaaaggcttcttcagctccaagagcaaatgcgagccctttataaagccatcagtgtccctcgagtccgcagggccagctccaagggtggtggaggctacacttgtcagagtggctctggctgggatgagttcaccaaacatgtcaccagtgaatgtttggggtggatgaggcagcaaagggcggagatggacatggtggcctggggtgtggacctggcctcagtggagcagcacattaacagccaccggggcatccacaactccatcggcgactatcgctggcagctggacaaaatcaaagccgacctgcgcgagaaatctgctatctaccagttggaggaggagtatgaaaacctgctgaaagcgtcctttgagaggatggatcacctgcgacagctgcagaacatcattcaggccacgtccagggagatcatgtggatcaatgactgcgaggaggaggagctgctgtacgactggagcgacaagaacaccaacatcgctcagaaacaggaggccttctccatacgcatgagtcaactggaagttaaagaaaaagagctcaataagctgaaacaagaaagtgaccaacttgtcctcaatcagcatccagcttcagacaaaattgaggcctatatggacactctgcagacgcagtggagttggattcttcagatcaccaagtgcattgatgttcatctgaaagaaaatgctgcctactttcagttttttgaagaggcgcagtctactgaagcatacctgaaggggctccaggactccatcaggaagaagtacccctgcgacaagaacatgcccctgcagcacctgctggaacagatcaaggagctggagaaagaacgagagaaaatccttgaatacaagcgtcaggtgcagaacttggtaaacaagtctaagaagattgtacagctgaagcctcgtaacccagactacagaagcaataaacccattattctcagagctctctgtgactacaaacaagatcagaaaatcgtgcataagggggatgagtgtatcctgaaggacaacaacgagcgcagcaagtggtacgtgacgggcccgggaggcgttgacatgcttgttccctctgtggggctgatcatccctcctccgaacccactggccgtggacctctcttgcaagattgagcagtactacgaagccatcttggctctgtggaaccagctctacatcaacatgaagagcctggtgtcctggcactactgcatgattgacatagagaagatcagggccatgacaatcgccaagctgaaaacaatgcggcaggaagattacatgaagacgatagccgaccttgagttacattaccaagagttcatcagaaatagccaaggctcagagatgtttggagatgatgacaagcggaaaatacagtctcagttcaccgatgcccagaagcattaccagaccctggtcattcagctccctggctatccccagcaccagacagtgaccacaactgaaatcactcatcatggaacctgccaagatgtcaaccataataaagtaattgaaaccaacagagaaaatgacaagcaagaaacatggatgctgatggagctgcagaagattcgcaggcagatagagcactgcgagggcaggatgactctcaaaaacctccctctagcagaccagggatcttctcaccacatcacagtgaaaattaacgagcttaagagtgtgcagaatgattcacaagcaattgctgaggttctcaaccagcttaaagatatgcttgccaacttcagaggttctgaaaagtactgctatttacagaatgaagtatttggactatttcagaaactggaaaatatcaatggtgttacagatggctacttaaatagcttatgcacagtaagggcactgctccaggctattctccaaacagaagacatgttaaaggtttatgaagccaggctcactgaggaggaaactgtctgcctggacctggataaagtggaagcttaccgctgtggactgaagaaaataaaaaatgacttgaacttgaagaagtcgttgttggccactatgaagacagaactacagaaagcccagcagatccactctcagacttcacagcagtatccactttatgatctggacttgggcaagttcggtgaaaaagtcacacagctgacagaccgctggcaaaggatagataaacagatcgactttaggttatgggacctggagaaacaaatcaagcaattgaggaattatcgtgataactatcaggctttctgcaagtggctctatgatgctaaacgccgccaggattccttagaatccatgaaatttggagattccaacacagtcatgcggtttttgaatgagcagaagaacttgcacagtgaaatatctggcaaacgagacaaatcagaggaagtacaaaaaattgctgaactttgcgccaattcaattaaggattatgagctccagctggcctcatacacctcaggactggaaactctgctgaacatacctatcaagaggaccatgattcagtccccttctggggtgattctgcaagaggctgcagatgttcatgctcggtacattgaactacttacaagatctggagactattacaggttcttaagtgagatgctgaagagtttggaagatctgaagctgaaaaataccaagatcgaagttttggaagaggagctcagactggcccgagatgccaactcggaaaactgtaataagaacaaattcctggatcagaacctgcagaaataccaggcagagtgttcccagttcaaagcgaagcttgcgagcctggaggagctgaagagacaggctgagctggatgggaagtcggctaagcaaaatctagacaagtgctacggccaaataaaagaactcaatgagaagatcacccgactgacttatgagattgaagatgaaaagagaagaagaaaatctgtggaagacagatttgaccaacagaagaatgactatgaccaactgcagaaagcaaggcaatgtgaaaaggagaaccttggttggcagaaattagagtctgagaaagccatcaaggagaaggagtacgagattgaaaggttgagggttctactgcaggaagaaggcacccggaagagagaatatgaaaatgagctggcaaaggtaagaaaccactataatgaggagatgagtaatttaaggaacaagtatgaaacagagattaacattacgaagaccaccatcaaggagatatccatgcaaaaagaggatgattccaaaaatcttagaaaccagcttgatagactttcaagggaaaatcgagatctgaaggatgaaattgtcaggctcaatgacagcatcttgcaggccactgagcagcgaaggcgagctgaagaaaacgcccttcagcaaaaggcctgtggctctgagataatgcagaagaagcagcatctggagatagaactgaagcaggtcatgcagcagcgctctgaggacaatgcccggcacaagcagtccctggaggaggctgccaagaccattcaggacaaaaataaggagatcgagagactcaaagctgagtttcaggaggaggccaagcgccgctgggaatatgaaaatgaactgagtaaggtaagaaacaattatgatgaggagatcattagcttaaaaaatcagtttgagaccgagatcaacatcaccaagaccaccatccaccagctcaccatgcagaaggaagaggataccagtggctaccgggctcagatagacaatctcacccgagaaaacaggagcttatctgaagaaataaagaggctgaagaacactctaacccagaccacagagaatctcaggagggtggaagaagacatccaacagcaaaaggccactggctctgaggtgtctcagaggaaacagcagctggaggttgagctgagacaagtcactcagatgcgaacagaggagagcgtaagatataagcaatctcttgatgatgctgccaaaaccatccaggataaaaacaaggagatagaaaggttaaaacaactgatcgacaaagaaacaaatgaccggaaatgcctggaagatgaaaacgcgagattacaaagggtccagtatgacctgcagaaagcaaacagtagtgcgacggagacaataaacaaactgaaggttcaggagcaagaactgacacgcctgaggatcgactatgaaagggtttcccaggagaggactgtgaaggaccaggatatcacgcggttccagaactctctgaaagagctgcagctgcagaagcagaaggtggaagaggagctgaatcggctgaagaggaccgcgtcagaagactcctgcaagaggaagaagctggaggaagagctggaaggcatgaggaggtcgctgaaggagcaagccatcaaaatcaccaacctgacccagcagctggagcaggcatccattgttaagaagaggagtgaggatgacctccggcagcagagggacgtgctggatggccacctgagggaaaagcagaggacccaggaagagctgaggaggctctcttctgaggtcgaggccctgaggcggcagttactccaggaacaggaaagtgtcaaacaagctcacttgaggaatgagcatttccagaaggcgatagaagataaaagcagaagcttaaatgaaagcaaaatagaaattgagaggctgcagtctctcacagagaacctgaccaaggagcacttgatgttagaagaagaactgcggaacctgaggctggagtacgatgacctgaggagaggacgaagcgaagcggacagtgataaaaatgcaaccatcttggaactaaggagccagctgcagatcagcaacaaccggaccctggaactgcaggggctgattaatgatttacagagagagagggaaaatttgagacaggaaattgagaaattccaaaagcaggctttagaggcatctaataggattcaggaatcaaagaatcagtgtactcaggtggtacaggaaagagagagccttctggtgaaaatcaaagtcctggagcaagacaaggcaaggctgcagaggctggaggatgagctgaatcgtgcaaaatcaactctagaggcagaaaccagggtgaaacagcgcctggagtgtgagaaacagcaaattcagaatgacctgaatcagtggaagactcaatattcccgcaaggaggaggctattaggaagatagaatcggaaagagaaaagagtgagagagagaagaacagtcttaggagtgagatcgaaagactccaagcagagatcaagagaattgaagagaggtgcaggcgtaagctggaggattctaccagggagacacagtcacagttagaaacagaacgctcccgatatcagagggagattgataaactcagacagcgcccatatgggtcccatcgagagacccagactgagtgtgagtggaccgttgacacctccaagctggtgtttgatgggctgaggaagaaggtgacagcaatgcagctctatgagtgtcagctgatcgacaaaacaaccttggacaaactattgaaggggaagaagtcagtggaagaagttgcttctgaaatccagccattccttcggggtgcaggatctatcgctggagcatctgcttctcctaaggaaaaatactctttggtagaggccaagagaaagaaattaatcagcccagaatccacagtcatgcttctggaggcccaggcagctacaggtggtataattgatccccatcggaatgagaagctgactgtcgacagtgccatagctcgggacctcattgacttcgatgaccgtcagcagatatatgcagcagaaaaagctatcactggttttgatgatccattttcaggcaagacagtatctgtttcagaagccatcaagaaaaatttgattgatagagaaaccggaatgcgcctgctggaagcccagattgcttcagggggtgtagtagaccctgtgaacagtgtctttttgccaaaagatgtcgccttggcccgggggctgattgatagagatttgtatcgatccctgaatgatccccgagatagtcagaaaaactttgtggatccagtcaccaaaaagaaggtcagttacgtgcagctgaaggaacggtgcagaatcgaaccacatactggtctgctcttgctttcagtacagaagagaagcatgtccttccaaggaatcagacaacctgtgaccgtcactgagctagtagattctggtatattgagaccgtccactgtcaatgaactggaatctggtcagatttcttatgacgaggttggtgagagaattaaggacttcctccagggttcaagctgcatagcaggcatatacaatgagaccacaaaacagaagcttggcatttatgaggccatgaaaattggcttagtccgacctggtactgctctggagttgctggaagcccaagcagctactggctttatagtggatcctgttagcaacttgaggttaccagtggaggaagcctacaagagaggtctggtgggcattgagttcaaagagaagctcctgtctgcagaacgagctgtcactgggtataatgatcctgaaacaggaaacatcatctctttgttccaagccatgaataaggaactcatcgaaaagggccacggtattcgcttattagaagcacagatcgcaaccggggggatcattgacccaaaggagagccatcgtttaccagttgacatagcatataagaggggctatttcaatgaggaactcagtgagattctctcagatccaagtgatgataccaaaggattttttgaccccaacactgaagaaaatcttacctatctgcaactaaaagaaagatgcattaaggatgaggaaacagggctctgtcttctgcctctgaaagaaaagaagaaacaggtgcagacatcacaaaagaataccctcaggaagcgtagagtggtcatagttgacccagaaaccaataaagaaatgtctgttcaggaggcctacaagaagggcctaattgattatgaaaccttcaaagaactgtgtgagcaggaatgtgaatgggaagaaataaccatcacgggatcagatggctccaccagggtggtcctggtagatagaaagacaggcagtcagtatgatattcaagatgctattgacaagggccttgttgacaggaagttctttgatcagtaccgatccggcagcctcagcctcactcaatttgctgacatgatctccttgaaaaatggtgtcggcaccagcagcagcatgggcagtggtgtcagcgatgatgtttttagcagctcccgacatgaatcagtaagtaagatttccaccatatccagcgtcaggaatttaaccataaggagcagctctttttcagacaccctggaagaatcgagccccattgcagccatctttgacacagaaaacctggagaaaatctccattacagaaggtatagagcggggcatcgttgacagcatcacgggtcagaggcttctggaggctcaggcctgcacaggtggcatcatccacccaaccacgggccagaagctgtcacttcaggacgcagtctcccagggtgtgattgaccaagacatggccaccaggctgaagcctgctcagaaagccttcataggcttcgagggtgtgaagggaaagaagaagatgtcagcagcagaggcagtgaaagaaaaatggctcccgtatgaggctggccagcgcttcctggagttccagtacctcacgggaggtcttgttgacccggaagtgcatgggaggataagcaccgaagaagccatccggaaggggttcatagatggccgcgccgcacagaggctgcaagacaccagcagctatgccaaaatcctgacctgccccaaaaccaaattaaaaatatcctataaggatgccataaatcgctccatggtagaagatatcactgggctgcgccttctggaagccgcctccgtgtcgtccaagggcttacccagcccttacaacatgtcttcggctccggggtcccgctccggctcccgctcgggatctcgctccggatctcgctccgggtcccgcagtgggtcccggagaggaagctttgacgccacagggaattcttcctactcttattcctactcatttagcagtagttctattgggcactag

SEQ ID NO: 26 - DNA, 5883 bp, Homo sapiens
atggcacagcaagctgccgataagtatctctatgtggataaaaacttcatcaacaatccgctggcccaggccgactgggctgccaagaagctggtatgggtgccttccgacaagagtggctttgagccagccagcctcaaggaggaggtgggcgaagaggccatcgtggagctggtggagaatgggaagaaggtgaaggtgaacaaggatgacatccagaagatgaacccgcccaagttctccaaggtggaggacatggcagagctcacgtgcctcaacgaagcctcggtgctgcacaacctcaaggagcgttactactcagggctcatctacacctattcaggcctgttctgtgtggtcatcaatccttacaagaacctgcccatctactctgaagagattgtggaaatgtacaagggcaagaagaggcacgagatgccccctcacatctatgccatcacagacaccgcctacaggagtatgatgcaagaccgagaagatcaatccatcttgtgcactggtgaatctggagctggcaagacggagaacaccaagaaggtcatccagtatctggcgtacgtggcgtcctcgcacaagagcaagaaggaccagggcgagctggagcggcagctgctgcaggccaaccccatcctggaggccttcgggaacgccaagaccgtgaagaatgacaactcctcccgcttcggcaaattcattcgcatcaactttgatgtcaatggctacattgttggagccaacattgagacttatcttttggagaaatctcgtgctatccgccaagccaaggaagaacggaccttccacatcttctattatctcctgtctggggctggagagcacctgaagaccgatctcctgttggagccgtacaacaaataccgcttcctgtccaatggacacgtcaccatccccgggcagcaggacaaggacatgttccaggagaccatggaggccatgaggattatgggcatcccagaagaggagcaaatgggcctgctgcgggtcatctcaggggttcttcagctcggcaacatcgtcttcaagaaggagcggaacactgaccaggcgtccatgcccgacaacacagctgcccaaaaggtgtcccatctcttgggtatcaatgtgaccgatttcaccagaggaatcctcaccccgcgcatcaaggtgggacgggattacgtccagaaggcgcagactaaagagcaggctgactttgccatcgaggccttggccaaggcgacctatgagcggatgttccgctggctggtgctgcgcatcaacaaggctctggacaagaccaagaggcagggcgcctccttcatcgggatcctggacattgccggcttcgagatctttgatctgaactcgtttgagcagctgtgcatcaattacaccaatgagaagctgcagcagctcttcaaccacaccatgttcatcctggagcaggaggagtaccagcgcgagggcatcgagtggaacttcatcgactttggcctcgacctgcagccctgcatcgacctcattgagaagccagcaggccccccgggcattctggccctgctggacgaggagtgctggttccccaaagccaccgacaagagcttcgtggagaaggtgatgcaggagcagggcacccaccccaagttccagaagcccaagcagctgaaggacaaagctgatttctgcattatccactatgccggcaaggtggattacaaagctgacgagtggctgatgaagaacatggatcccctgaatgacaacatcgccacactgctccaccagtcctctgacaagtttgtctcggagctgtggaaggatgtggaccgcatcatcggcctggaccaggtggccggcatgtcggagaccgcactgcccggggccttcaagacgcggaagggcatgttccgcactgtggggcagctttacaaggagcagctggccaagctgatggctacgctgaggaacacgaaccccaactttgtccgctgcatcatccccaaccacgagaagaaggccggcaagctggacccgcatctcgtgctggaccagctgcgctgcaacggtgttctcgagggcatccgtatctgccgccagggcttccccaacagggtggtcttccaggagtttcggcagagatatgagatcctgactccaaactccattcccaagggtttcatggacgggaagcaggcgtgcgtgctcatgataaaagccctggagctcgacagcaatctgtaccgcattggccagagcaaagtcttcttccgtgccggtgtgctggcccacctggaggaggagcgagacctgaagatcaccgacgtcatcatagggttccaggcctgctgcaggggctacctggccaggaaagcatttgccaagcggcagcagcagcttaccgccatgaaggtcctccagcggaactgcgctgcctacctgaagctgcggaactggcagtggtggcggctcttcaccaaggtcaagccgctgctgcaggtgagccggcaggaggaggagatgatggccaaggaggaggagctggtgaaggtcagagagaagcagctggctgcggagaacaggctcacggagatggagacgctgcagtctcagctcatggcagagaaattgcagctgcaggagcagctccaggcagaaaccgagctgtgtgccgaggctgaggagctccgggcccgcctgaccgccaagaagcaggaattagaagagatctgccatgacctagaggccagggtggaggaggaggaggagcgctgccagcacctgcaggcggagaagaagaagatgcagcagaacatccaggagcttgaggagcagctggaggaggaggagagcgcccggcagaagctgcagctggagaaggtgaccaccgaggcgaagctgaaaaagctggaggaggagcagatcatcctggaggaccagaactgcaagctggccaaggaaaagaaactgctggaagacagaatagctgagttcaccaccaacctcacagaagaggaggagaaatctaagagcctcgccaagctcaagaacaagcatgaggcaatgatcactgacttggaagagcgcctccgcagggaggagaagcagcgacaggagctggagaagacccgccggaagctggagggagactccacagacctcagcgaccagatcgccgagctccaggcccagatcgcggagctcaagatgcagctggccaagaaagaggaggagctccaggccgccctggccagagtggaagaggaagctgcccagaagaacatggccctcaagaagatccgggagctggaatctcagatctctgaactccaggaagacctggagtctgagcgtgcttccaggaataaagctgagaagcagaaacgggaccttggggaagagctagaggctctgaaaacagagttggaggacacgctggattccacagctgcccagcaggagctcaggtcaaaacgtgagcaggaggtgaacatcctgaagaagaccctggaggaggaggccaagacccacgaggcccagatccaggagatgaggcagaagcactcacaggccgtggaggagctggcggagcagctggagcagacgaagcgggtgaaagcaaacctcgagaaggcaaagcagactctggagaacgagcggggggagctggccaacgaggtgaaggtgctgctgcagggcaaaggggactcggagcacaagcgcaagaaagtggaggcgcagctgcaggagctgcaggtcaagttcaacgagggagagcgcgtgcgcacagagctggccgacaaggtcaccaagctgcaggtggagctggacaacgtgaccgggcttctcagccagtccgacagcaagtccagcaagctcaccaaggacttctccgcgctggagtcccagctgcaggacactcaggagctgctgcaggaggagaaccggcagaagctgagcctgagcaccaagctcaagcaggtggaggacgagaagaattccttccgggagcagctggaggaggaggaggaggccaagcacaacctggagaagcagatcgccaccctccatgcccaggtggccgacatgaaaaagaagatggaggacagtgtggggtgcctggaaactgctgaggaggtgaagaggaagctccagaaggacctggagggcctgagccagcggcacgaggagaaggtggccgcctacgacaagctggagaagaccaagacgcggctgcagcaggagctggacgacctgctggtggacctggaccaccagcgccagagcgcgtgcaacctggagaagaagcagaagaagtttgaccagctcctggcggaggagaagaccatctctgccaagtatgcagaggagcgcgaccgggctgaggcggaggcccgagagaaggagaccaaggctctgtcgctggcccgggccctggaggaagccatggagcagaaggcggagctggagcggctcaacaagcagttccgcacggagatggaggaccttatgagctccaaggatgatgtgggcaagagtgtccacgagctggagaagtccaagcgggccctagagcagcaggtggaggagatgaagacgcagctggaagagctggaggacgagctgcaggccaccgaagatgccaagctgcggttggaggtcaacctgcaggccatgaaggcccagttcgagcgggacctgcagggccgggacgagcagagcgaggagaagaagaagcagctggtcagacaggtgcgggagatggaggcagagctggaggacgagaggaagcagcgctcgatggcagtggccgcccggaagaagctggagatggacctgaaggacctggaggcgcacatcgactcggccaacaagaaccgggacgaagccatcaaacagctgcggaagctgcaggcccagatgaaggactgcatgcgcgagctggatgacacccgcgcctctcgtgaggagatcctggcccaggccaaagagaacgagaagaagctgaagagcatggaggccgagatgatccagttgcaggaggaactggcagccgcggagcgtgccaagcgccaggcccagcaggagcgggatgagctggctgacgagatcgccaacagcagcggcaaaggagccctggcgttagaggagaagcggcgtctggaggcccgcatcgcccagctggaggaggagctggaggaggagcagggcaacacggagctgatcaacgaccggctgaagaaggccaacctgcagatcgaccagatcaacaccgacctgaacctggagcgcagccacgcccagaagaacgagaatgctcggcagcagctggaacgccagaacaaggagcttaaggtcaagctgcaggagatggagggcactgtcaagtccaagtacaaggcctccatcaccgccctcgaggccaagattgcacagctggaggagcagctggacaacgagaccaaggagcgccaggcagcctgcaaacaggtgcgtcggaccgagaagaagctgaaggatgtgctgctgcaggtggatgacgagcggaggaacgccgagcagtacaaggaccaggccgacaaggcatctacccgcctgaagcagctcaagcggcagctggaggaggccgaagaggaggcccagcgggccaacgcctcccgccggaaactgcagcgcgagctggaggacgccactgagacggccgatgccatgaaccgcgaagtcagctccctaaagaacaagctcaggcgcggggacctgccgtttgtcgtgccccgccgaatggcccggaaaggcgccggggatggctccgacgaagaggtagatggcaaagcggatggggctgaggccaaacctgccgaataa

SEQ ID NO: 27 - DNA, 747 bp, Homo sapiens
atggagagagccagtctgatccagaaggccaagctggcagagcaggccgaacgctatgaggacatggcagccttcatgaaaggcgccgtggagaagggcgaggagctctcctgcgaagagcgaaacctgctctcagtagcctataagaacgtggtgggcggccagagggctgcctggagggtgctgtccagtattgagcagaaaagcaacgaggagggctcggaggagaaggggcccgaggtgcgtgagtaccgggagaaggtggagactgagctccagggcgtgtgcgacaccgtgctgggcctgctggacagccacctcatcaaggaggccggggacgccgagagccgggtcttctacctgaagatgaagggtgactactaccgctacctggccgaggtggccaccggtgacgacaagaagcgcatcattgactcagcccggtcagcctaccaggaggccatggacatcagcaagaaggagatgccgcccaccaaccccatccgcctgggcctggccctgaacttttccgtcttccactacgagatcgccaacagccccgaggaggccatctctctggccaagaccactttcgacgaggccatggctgatctgcacaccctcagcgaggactcctacaaagacagcaccctcatcatgcagctgctgcgagacaacctgacactgtggacggccgacaacgccggggaagaggggggcgaggctccccaggagccccagagctga

SEQ ID NO: 28 - protein, 2871 aa, Homo sapiens
MSCNGGSHPRINTLGRMIRAESGPDLRYEVTSGGGGTSRMYYSRRGVITDQNSDGYCQTGTMSRHQNQNTIQELLQNCSDCLMRAELIVQPELKYGDGIQLTRSRELDECFAQANDQMEILDSLIREMRQMGQPCDAYQKRLLQLQEQMRALYKAISVPRVRRASSKGGGGYTCQSGSGWDEFTKHVTSECLGWMRQQRAEMDMVAWGVDLASVEQHINSHRGIHNSIGDYRWQLDKIKADLREKSAIYQLEEEYENLLKASFERMDHLRQLQNIIQATSREIMWINDCEEEELLYDWSDKNTNIAQKQEAFSIRMSQLEVKEKELNKLKQESDQLVLNQHPASDKIEAYMDTLQTQWSWILQITKCIDVHLKENAAYFQFFEEAQSTEAYLKGLQDSIRKKYPCDKNMPLQHLLEQIKELEKEREKILEYKRQVQNLVNKSKKIVQLKPRNPDYRSNKPIILRALCDYKQDQKIVHKGDECILKDNNERSKWYVTGPGGVDMLVPSVGLIIPPPNPLAVDLSCKIEQYYEAILALWNQLYINMKSLVSWHYCMIDIEKIRAMTIAKLKTMRQEDYMKTIADLELHYQEFIRNSQGSEMFGDDDKRKIQSQFTDAQKHYQTLVIQLPGYPQHQTVTTTEITHHGTCQDVNHNKVIETNRENDKQETWMLMELQKIRRQIEHCEGRMTLKNLPLADQGSSHHITVKINELKSVQNDSQAIAEVLNQLKDMLANFRGSEKYCYLQNEVFGLFQKLENINGVTDGYLNSLCTVRALLQAILQTEDMLKVYEARLTEEETVCLDLDKVEAYRCGLKKIKNDLNLKKSLLATMKTELQKAQQIHSQTSQQYPLYDLDLGKFGEKVTQLTDRWQRIDKQIDFRLWDLEKQIKQLRNYRDNYQAFCKWLYDAKRRQDSLESMKFGDSNTVMRFLNEQKNLHSEISGKRDKSEEVQKIAELCANSIKDYELQLASYTSGLETLLNIPIKRTMIQSPSGVILQEAADVHARYIELLTRSGDYYRFLSEMLKSLEDLKLKNTKIEVLEEELRLARDANSENCNKNKFLDQNLQKYQAECSQFKAKLASLEELKRQAELDGKSAKQNLDKCYGQIKELNEKITRLTYEIEDEKRRRKSVEDRFDQQKNDYDQLQKARQCEKENLGWQKLESEKAIKEKEYEIERLRVLLQEEGTRKREYENELAKVRNHYNEEMSNLRNKYETEINITKTTIKEISMQKEDDSKNLRNQLDRLSRENRDLKDEIVRLNDSILQATEQRRRAEENALQQKACGSEIMQKKQHLEIELKQVMQQRSEDNARHKQSLEEAAKTIQDKNKEIERLKAEFQEEAKRRWEYENELSKVRNNYDEEIISLKNQFETEINITKTTIHQLTMQKEEDTSGYRAQIDNLTRENRSLSEEIKRLKNTLTQTTENLRRVEEDIQQQKATGSEVSQRKQQLEVELRQVTQMRTEESVRYKQSLDDAAKTIQDKNKEIERLKQLIDKETNDRKCLEDENARLQRVQYDLQKANSSATETINKLKVQEQELTRLRIDYERVSQERTVKDQDITRFQNSLKELQLQKQKVEEELNRLKRTASEDSCKRKKLEEELEGMRRSLKEQAIKITNLTQQLEQASIVKKRSEDDLRQQRDVLDGHLREKQRTQEELRRLSSEVEALRRQLLQEQESVKQAHLRNEHFQKAIEDKSRSLNESKIEIERLQSLTENLTKEHLMLEEELRNLRLEYDDLRRGRSEADSDKNATILELRSQLQISNNRTLELQGLINDLQRERENLRQEIEKFQKQALEASNRIQESKNQCTQVVQERESLLVKIKVLEQDKARLQRLEDELNRAKSTLEAETRVKQRLECEKQQIQNDLNQWKTQYSRKEEAIRKIESEREKSEREKNSLRSEIERLQAEIKRIEERCRRKLEDSTRETQSQLETERSRYQREIDKLRQRPYGSHRETQTECEWTVDTSKLVFDGLRKKVTAMQLYECQLIDKTTLDKLLKGKKSVEEVASEIQPFLRGAGSIAGASASPKEKYSLVEAKRKKLISPESTVMLLEAQAATGGIIDPHRNEKLTVDSAIARDLIDFDDRQQIYAAEKAITGFDDPFSGKTVSVSEAIKKNLIDRETGMRLLEAQIASGGVVDPVNSVFLPKDVALARGLIDRDLYRSLNDPRDSQKNFVDPVTKKKVSYVQLKERCRIEPHTGLLLLSVQKRSMSFQGIRQPVTVTELVDSGILRPSTVNELESGQISYDEVGERIKDFLQGSSCIAGIYNETTKQKLGIYEAMKIGLVRPGTALELLEAQAATGFIVDPVSNLRLPVEEAYKRGLVGIEFKEKLLSAERAVTGYNDPETGNIISLFQAMNKELIEKGHGIRLLEAQIATGGIIDPKESHRLPVDIAYKRGYFNEELSEILSDPSDDTKGFFDPNTEENLTYLQLKERCIKDEETGLCLLPLKEKKKQVQTSQKNTLRKRRVVIVDPETNKEMSVQEAYKKGLIDYETFKELCEQECEWEEITITGSDGSTRVVLVDRKTGSQYDIQDAIDKGLVDRKFFDQYRSGSLSLTQFADMISLKNGVGTSSSMGSGVSDDVFSSSRHESVSKISTISSVRNLTIRSSSFSDTLEESSPIAAIFDTENLEKISITEGIERGIVDSITGQRLLEAQACTGGIIHPTTGQKLSLQDAVSQGVIDQDMATRLKPAQKAFIGFEGVKGKKKMSAAEAVKEKWLPYEAGQRFLEFQYLTGGLVDPEVHGRISTEEAIRKGFIDGRAAQRLQDTSSYAKILTCPKTKLKISYKDAINRSMVEDITGLRLLEAASVSSKGLPSPYNMSSAPGSRSGSRSGSRSGSRSGSRSGSRRGSFDATGNSSYSYSYSFSSSSIGH

SEQ ID NO: 29 - protein, 1960 aa, Homo sapiens
MAQQAADKYLYVDKNFINNPLAQADWAAKKLVWVPSDKSGFEPASLKEEVGEEAIVELVENGKKVKVNKDDIQKMNPPKFSKVEDMAELTCLNEASVLHNLKERYYSGLIYTYSGLFCVVINPYKNLPIYSEEIVEMYKGKKRHEMPPHIYAITDTAYRSMMQDREDQSILCTGESGAGKTENTKKVIQYLAYVASSHKSKKDQGELERQLLQANPILEAFGNAKTVKNDNSSRFGKFIRINFDVNGYIVGANIETYLLEKSRAIRQAKEERTFHIFYYLLSGAGEHLKTDLLLEPYNKYRFLSNGHVTIPGQQDKDMFQETMEAMRIMGIPEEEQMGLLRVISGVLQLGNIVFKKERNTDQASMPDNTAAQKVSHLLGINVTDFTRGILTPRIKVGRDYVQKAQTKEQADFAIEALAKATYERMFRWLVLRINKALDKTKRQGASFIGILDIAGFEIFDLNSFEQLCINYTNEKLQQLFNHTMFILEQEEYQREGIEWNFIDFGLDLQPCIDLIEKPAGPPGILALLDEECWFPKATDKSFVEKVMQEQGTHPKFQKPKQLKDKADFCIIHYAGKVDYKADEWLMKNMDPLNDNIATLLHQSSDKFVSELWKDVDRIIGLDQVAGMSETALPGAFKTRKGMFRTVGQLYKEQLAKLMATLRNTNPNFVRCIIPNHEKKAGKLDPHLVLDQLRCNGVLEGIRICRQGFPNRVVFQEFRQRYEILTPNSIPKGFMDGKQACVLMIKALELDSNLYRIGQSKVFFRAGVLAHLEEERDLKITDVIIGFQACCRGYLARKAFAKRQQQLTAMKVLQRNCAAYLKLRNWQWWRLFTKVKPLLQVSRQEEEMMAKEEELVKVREKQLAAENRLTEMETLQSQLMAEKLQLQEQLQAETELCAEAEELRARLTAKKQELEEICHDLEARVEEEEERCQHLQAEKKKMQQNIQELEEQLEEEESARQKLQLEKVTTEAKLKKLEEEQIILEDQNCKLAKEKKLLEDRIAEFTTNLTEEEEKSKSLAKLKNKHEAMITDLEERLRREEKQRQELEKTRRKLEGDSTDLSDQIAELQAQIAELKMQLAKKEEELQAALARVEEEAAQKNMALKKIRELESQISELQEDLESERASRNKAEKQKRDLGEELEALKTELEDTLDSTAAQQELRSKREQEVNILKKTLEEEAKTHEAQIQEMRQKHSQAVEELAEQLEQTKRVKANLEKAKQTLENERGELANEVKVLLQGKGDSEHKRKKVEAQLQELQVKFNEGERVRTELADKVTKLQVELDNVTGLLSQSDSKSSKLTKDFSALESQLQDTQELLQEENRQKLSLSTKLKQVEDEKNSFREQLEEEEEAKHNLEKQIATLHAQVADMKKKMEDSVGCLETAEEVKRKLQKDLEGLSQRHEEKVAAYDKLEKTKTRLQQELDDLLVDLDHQRQSACNLEKKQKKFDQLLAEEKTISAKYAEERDRAEAEAREKETKALSLARALEEAMEQKAELERLNKQFRTEMEDLMSSKDDVGKSVHELEKSKRALEQQVEEMKTQLEELEDELQATEDAKLRLEVNLQAMKAQFERDLQGRDEQSEEKKKQLVRQVREMEAELEDERKQRSMAVAARKKLEMDLKDLEAHIDSANKNRDEAIKQLRKLQAQMKDCMRELDDTRASREEILAQAKENEKKLKSMEAEMIQLQEELAAAERAKRQAQQERDELADEIANSSGKGALALEEKRRLEARIAQLEEELEEEQGNTELINDRLKKANLQIDQINTDLNLERSHAQKNENARQQLERQNKELKVKLQEMEGTVKSKYKASITALEAKIAQLEEQLDNETKERQAACKQVRRTEKKLKDVLLQVDDERRNAEQYKDQADKASTRLKQLKRQLEEAEEEAQRANASRRKLQRELEDATETADAMNREVSSLKNKLRRGDLPFVVPRRMARKGAGDGSDEEVDGKADGAEAKPAE

SEQ ID NO: 30 - protein, 248 aa, Homo sapiens [sequence omitted]

SEQ ID NO: 31 - DNA, 1410 bp, Homo sapiens
atgtccatccacttcagctccccggtattcacctcgcgctcagccgccttctcgggccgcggcgcccaggtgcgcctgagctccgctcgccccggcggccttggcagcagcagcctctacggcctcggcgcctcacggccgcgcgtggccgtgcgctctgcctatgggggcccggtgggcgccggcatccgcgaggtcaccattaaccagagcctgctggccccgctgcggctggacgccgacccctccctccagcgggtgcgccaggaggagagcgagcagatcaagaccctcaacaacaagtttgcctccttcatcgacaaggtgcggtttctggagcagcagaacaagctgctggagaccaagtggacgctgctgcaggagcagaagtcggccaagagcagccgcctcccagacatctttgaggcccagattgctggccttcggggtcagcttgaggcactgcaggtggatgggggccgcctggaggcggagctgcggagcatgcaggatgtggtggaggacttcaagaataagtacgaagatgaaattaaccaccgcacagctgctgagaatgagtttgtggtgctgaagaaggatgtggatgctgcctacatgagcaaggtggagctggaggccaaggtggatgccctgaatgatgagatcaacttcctcaggaccctcaatgagacggagttgacagagctgcagtcccagatctccgacacatctgtggtgctgtccatggacaacagtcgctccctggacctggacggcatcatcgctgaggtcaaggcgcagtatgaggagatggccaaatgcagccgggctgaggctgaagcctggtaccagaccaagtttgagaccctccaggcccaggctgggaagcatggggacgacctccggaatacccggaatgagatttcagagatgaaccgggccatccagaggctgcaggctgagatcgacaacatcaagaaccagcgtgccaagttggaggccgccattgccgaggctgaggagcgtggggagctggcgctcaaggatgctcgtgccaagcaggaggagctggaagccgccctgcagcggggcaagcaggatatggcacggcagctgcgtgagtaccaggaactcatgagcgtgaagctggccctggacatcgagatcgccacctaccgcaagctgctggagggcgaggagagccggttggctggagatggagtgggagccgtgaatatctctgtgatgaattccactggtggcagtagcagtggcggtggcattgggctgaccctcgggggaaccatgggcagcaatgccctgagcttctccagcagtgcgggtcctgggctcctgaaggcttattccatccggaccgcatccgccagtcgcaggagtgcccgcgactga

SEQ ID NO: 32 - protein, 469 aa, Homo sapiens
MSIHFSSPVFTSRSAAFSGRGAQVRLSSARPGGLGSSSLYGLGASRPRVAVRSAYGGPVGAGIREVTINQSLLAPLRLDADPSLQRVRQEESEQIKTLNNKFASFIDKVRFLEQQNKLLETKWTLLQEQKSAKSSRLPDIFEAQIAGLRGQLEALQVDGGRLEAELRSMQDVVEDFKNKYEDEINHRTAAENEFVVLKKDVDAAYMSKVELEAKVDALNDEINFLRTLNETELTELQSQISDTSVVLSMDNSRSLDLDGIIAEVKAQYEEMAKCSRAEAEAWYQTKFETLQAQAGKHGDDLRNTRNEISEMNRAIQRLQAEIDNIKNQRAKLEAAIAEAEERGELALKDARAKQEELEAALQRGKQDMARQLREYQELMSVKLALDIEIATYRKLLEGEESRLAGDGVGAVNISVMNSTGGSSSGGGIGLTLGGTMGSNALSFSSSAGPGLLKAYSIRTASASRRSARD

SEQ ID NO: 33 - DNA, 1452 bp, Homo sapiens
atgtccatcagggtgacccagaagtcctacaaggtgtccacctctggcccccgggccttcagcagccgctcctacacgagtgggcccggttcccgcatcagctcctcgagcttctcccgagtgggcagcagcaactttcgcggtggcctgggcggcggctatggtggggccagcggcatgggaggcatcaccgcagttacggtcaaccagagcctgctgagcccccttgtcctggaggtggaccccaacatccaggccgtgcgcacccaggagaaggagcagatcaagaccctcaacaacaagtttgcctccttcatagacaaggtacggttcctggagcagcagaacaagatgctggagaccaagtggagcctcctgcagcagcagaagacggctcgaagcaacatggacaacatgttcgagagctacatcaacaaccttaggcggcagctggagactctgggccaggagaagctgaagctggaggcggagcttggcaacatgcaggggctggtggaggacttcaagaacaagtatgaggatgagatcaataagcgtacagagatggagaacgaatttgtcctcatcaagaaggatgtggatgaagcttacatgaacaaggtagagctggagtctcgcctggaagggctgaccgacgagatcaacttcctcaggcagctatatgaagaggagatccgggagctgcagtcccagatctcggacacatctgtggtgctgtccatggacaacagccgctccctggacatggacagcatcattgctgaggtcaaggcacagtacgaggatattgccaaccgcagccgggctgaggctgagagcatgtaccagatcaagtatgaggagctgcagagcctggctgggaagcacggggatgacctgcggcgcacaaagactgagatctctgagatgaaccggaacatcagccggctccaggctgagattgagggcctcaaaggccagagggcttccctggaggccgccattgcagatgccgagcagcgtggagagctggccattaaggatgccaacgccaagttgtccgagctggaggccgccctgcagcgggccaagcaggacatggcgcggcagctgcgtgagtaccaggagctgatgaacgtcaagctggccctggacatcgagatcgccacctacaggaagctgctggagggcgaggagagccggctggagtctgggatgcagaacatgagtattcatacgaagaccaccagcggctatgcaggtggtctgagctcggcctatgggggcctcacaagccccggcctcagctacagcctgggctccagctttggctctggcgcgggctccagctccttcagccgcaccagctcctccagggccgtggttgtgaagaagatcgagacacgtgatgggaagctggtgtctgagtcctctgacgtcctgcccaagtga

SEQ ID NO: 34 - protein, 483 aa, Homo sapiens
MSIRVTQKSYKVSTSGPRAFSSRSYTSGPGSRISSSSFSRVGSSNFRGGLGGGYGGASGMGGITAVTVNQSLLSPLVLEVDPNIQAVRTQEKEQIKTLNNKFASFIDKVRFLEQQNKMLETKWSLLQQQKTARSNMDNMFESYINNLRRQLETLGQEKLKLEAELGNMQGLVEDFKNKYEDEINKRTEMENEFVLIKKDVDEAYMNKVELESRLEGLTDEINFLRQLYEEEIRELQSQISDTSVVLSMDNSRSLDMDSIIAEVKAQYEDIANRSRAEAESMYQIKYEELQSLAGKHGDDLRRTKTEISEMNRNISRLQAEIEGLKGQRASLEAAIADAEQRGELAIKDANAKLSELEAALQRAKQDMARQLREYQELMNVKLALDIEIATYRKLLEGEESRLESGMQNMSIHTKTTSGYAGGLSSAYGGLTSPGLSYSLGSSFGSGAGSSSFSRTSSSRAVVVKKIETRDGKLVSESSDVLPK

SEQ ID NO: 35 - DNA, 1293 bp, Homo sapiens
atgagcttcaccactcgctccaccttctccaccaactaccggtccctgggctctgtccaggcgcccagctacggcgcccggccggtcagcagcgcggccagcgtctatgcaggcgctgggggctctggttcccggatctccgtgtcccgctccaccagcttcaggggcggcatggggtccgggggcctggccaccgggatagccgggggtctggcaggaatgggaggcatccagaacgagaaggagaccatgcaaagcctgaacgaccgcctggcctcttacctggacagagtgaggagcctggagaccgagaaccggaggctggagagcaaaatccgggagcacttggagaagaagggaccccaggtcagagactggagccattacttcaagatcatcgaggacctgagggctcagatcttcgcaaatactgtggacaatgcccgcatcgttctgcagattgacaatgcccgtcttgctgctgatgactttagagtcaagtatgagacagagctggccatgcgccagtctgtggagaacgacatccatgggctccgcaaggtcattgatgacaccaatatcacacgactgcagctggagacagagatcgaggctctcaaggaggagctgctcttcatgaagaagaaccacgaagaggaagtaaaaggcctacaagcccagattgccagctctgggttgaccgtggaggtagatgcccccaaatctcaggacctcgccaagatcatggcagacatccgggcccaatatgacgagctggctcggaagaaccgagaggagctagacaagtactggtctcagcagattgaggagagcaccacagtggtcaccacacagtctgctgaggttggagctgctgagacgacgctcacagagctgagacgtacagtccagtccttggagatcgacctggactccatgagaaatctgaaggccagcttggagaacagcctgagggaggtggaggcccgctacgccctacagatggagcagctcaacgggatcctgctgcaccttgagtcagagctggcacagacccgggcagagggacagcgccaggcccaggagtatgaggccctgctgaacatcaaggtcaagctggaggctgagatcgccacctaccgccgcctgctggaagatggcgaggactttaatcttggtgatgccttggacagcagcaactccatgcaaaccatccaaaagaccaccacccgccggatagtggatggcaaagtggtgtctgagaccaatgacaccaaagttctgaggcattaa

SEQ ID NO: 36 - protein, 430 aa, Homo sapiens
MSFTTRSTFSTNYRSLGSVQAPSYGARPVSSAASVYAGAGGSGSRISVSRSTSFRGGMGSGGLATGIAGGLAGMGGIQNEKETMQSLNDRLASYLDRVRSLETENRRLESKIREHLEKKGPQVRDWSHYFKIIEDLRAQIFANTVDNARIVLQIDNARLAADDFRVKYETELAMRQSVENDIHGLRKVIDDTNITRLQLETEIEALKEELLFMKKNHEEEVKGLQAQIASSGLTVEVDAPKSQDLAKIMADIRAQYDELARKNREELDKYWSQQIEESTTVVTTQSAEVGAAETTLTELRRTVQSLEIDLDSMRNLKASLENSLREVEARYALQMEQLNGILLHLESELAQTRAEGQRQAQEYEALLNIKVKLEAEIATYRRLLEDGEDFNLGDALDSSNSMQTIQKTTTRRIVDGKVVSETNDTKVLRH

SEQ ID NO: 37 - DNA, 1203 bp, Homo sapiens
atgacttcctacagctatcgccagtcgtcggccacgtcgtccttcggaggcctgggcggcggctccgtgcgttttgggccgggggtcgcctttcgcgcgcccagcattcacgggggctccggcggccgcggcgtatccgtgtcctccgcccgctttgtgtcctcgtcctcctcgggggcctacggcggcggctacggcggcgtcctgaccgcgtccgacgggctgctggcgggcaacgagaagctaaccatgcagaacctcaacgaccgcctggcctcctacctggacaaggtgcgcgccctggaggcggccaacggcgagctagaggtgaagatccgcgactggtaccagaagcaggggcctgggccctcccgcgactacagccactactacacgaccatccaggacctgcgggacaagattcttggtgccaccattgagaactccaggattgtcctgcagatcgacaatgcccgtctggctgcagatgacttccgaaccaagtttgagacggaacaggctctgcgcatgagcgtggaggccgacatcaacggcctgcgcagggtgctggatgagctgaccctggccaggaccgacctggagatgcagatcgaaggcctgaaggaagagctggcctacctgaagaagaaccatgaggaggaaatcagtacgctgaggggccaagtgggaggccaggtcagtgtggaggtggattccgctccgggcaccgatctcgccaagatcctgagtgacatgcgaagccaatatgaggtcatggccgagcagaaccggaaggatgctgaagcctggttcaccagccggactgaagaattgaaccgggaggtcgctggccacacggagcagctccagatgagcaggtccgaggttactgacctgcggcgcacccttcagggtcttgagattgagctgcagtcacagctgagcatgaaagctgccttggaagacacactggcagaaacggaggcgcgctttggagcccagctggcgcatatccaggcgctgatcagcggtattgaagcccagctgggcgatgtgcgagctgatagtgagcggcagaatcaggagtaccagcggctcatggacatcaagtcgcggctggagcaggagattgccacctaccgcagcctgctcgagggacaggaagatcactacaacaatttgtctgcctccaaggtcctctga

SEQ ID NO: 38 - protein, 400 aa, Homo sapiens
MTSYSYRQSSATSSFGGLGGGSVRFGPGVAFRAPSIHGGSGGRGVSVSSARFVSSSSSGAYGGGYGGVLTASDGLLAGNEKLTMQNLNDRLASYLDKVRALEAANGELEVKIRDWYQKQGPGPSRDYSHYYTTIQDLRDKILGATIENSRIVLQIDNARLAADDFRTKFETEQALRMSVEADINGLRRVLDELTLARTDLEMQIEGLKEELAYLKKNHEEEISTLRGQVGGQVSVEVDSAPGTDLAKILSDMRSQYEVMAEQNRKDAEAWFTSRTEELNREVAGHTEQLQMSRSEVTDLRRTLQGLEIELQSQLSMKAALEDTLAETEARFGAQLAHIQALISGIEAQLGDVRADSERQNQEYQRLMDIKSRLEQEIATYRSLLEGQEDHYNNLSASKVL

SEQ ID NO: 39 - DNA, 1275 bp, Homo sapiens
atggatttcagtcgcagaagcttccacagaagcctgagctcctccttgcaggcccctgtagtcagtacagtgggcatgcagcgcctcgggacgacacccagcgtttatgggggtgctggaggccggggcatccgcatctccaactccagacacacggtgaactatgggagcgatctcacaggcggcggggacctgtttgttggcaatgagaaaatggccatgcagaacctaaatgaccgtctagcgagctacctagaaaaggtgcggaccctggagcagtccaactccaaacttgaagtgcaaatcaagcagtggtacgaaaccaacgccccgagggctggtcgcgactacagtgcatattacagacaaattgaagagctgcgaagtcagattaaggatgctcaactgcaaaatgctcggtgtgtcctgcaaattgataatgctaaactggctgctgaggacttcagactgaagtatgagactgagagaggaatacgtctaacagtggaagctgatctccaaggcctgaataaggtctttgatgacctaaccctacataaaacagatttggagattcaaattgaagaactgaataaagacctagctctcctcaaaaaggagcatcaggaggaagtcgatggcctacacaagcatctgggcaacactgtcaatgtggaggttgatgctgctccaggcctgaaccttggcgtcatcatgaatgaaatgaggcagaagtatgaagtcatggcccagaagaaccttcaagaggccaaagaacagtttgagagacagactgcagttctgcagcaacaggtcacagtgaatactgaagaattaaaaggaactgaggttcaactaacggagctgagacgcacctcccagagccttgagatagaactccagtcccatctcagcatgaaagagtctttggagcacactctagaggagaccaaggcccgttacagcagccagttagccaacctccagtcgctgttgagctctctggaggcccaactgatgcagattcggagtaacatggaacgccagaacaacgaataccatatccttcttgacataaagactcgacttgaacaggaaattgctacttaccgccgccttctggaaggagaagacgtaaaaactacagaatatcagttaagcaccctggaagagagagatataaagaaaaccaggaagattaagacagtcgtgcaagaagtagtggatggcaaggtcgtgtcatctgaagtcaaagaggtggaagaaaatatctaa

SEQ ID NO: 40 - protein, 424 aa, Homo sapiens
MDFSRRSFHRSLSSSLQAPVVSTVGMQRLGTTPSVYGGAGGRGIRISNSRHTVNYGSDLTGGGDLFVGNEKMAMQNLNDRLASYLEKVRTLEQSNSKLEVQIKQWYETNAPRAGRDYSAYYRQIEELRSQIKDAQLQNARCVLQIDNAKLAAEDFRLKYETERGIRLTVEADLQGLNKVFDDLTLHKTDLEIQIEELNKDLALLKKEHQEEVDGLHKHLGNTVNVEVDAAPGLNLGVIMNEMRQKYEVMAQKNLQEAKEQFERQTAVLQQQVTVNTEELKGTEVQLTELRRTSQSLEIELQSHLSMKESLEHTLEETKARYSSQLANLQSLLSSLEAQLMQIRSNMERQNNEYHILLDIKTRLEQEIATYRRLLEGEDVKTTEYQLSTLEERDIKKTRKIKTVVQEVVDGKVVSSEVKEVEENI

SEQ ID NO: 41 - DNA, 22 bp, Homo sapiens
atggagtccagggatgaagtgt

SEQ ID NO: 42 - DNA, 22 bp, Homo sapiens
gatttggcaagcagcacataag

SEQ ID NO: 43 - DNA, 22 bp, Homo sapiens
ctctgctggacgtgtgctttac

SEQ ID NO: 44 - DNA, 22 bp, Homo sapiens
actcagggaccaaatccacagt